(19) United States
(12) Patent Application Publication
(10) Pub. No.: US 2005/0074743 A1
(43) Pub. Date: Apr. 7, 2005

(54) Method and composition for treating a biological sample

(76) Inventors: Andrei A. Purmal, Waltham, MA (US); John Chapman, Newton, MA (US); Jodie Tassello, Boston, MA (US); Dimitry Kamen, Nahant, MA (US); C. Robert Valeri, Marblehead, MA (US)

Correspondence Address: John R. Van Amsterdam, Ph.D.;Wolf, Greenfield & Sacks, P.C., 600 Atlantic Avenue, Boston, MA, 02210, US

(21) Appl. No.: 10/679,932

(22) Filed: Oct. 6, 2003

(51) Int. Cl. A01N 1/00; A61K 31/7076

(52) U.S. Cl. 435/1.1; 514/46

(57) ABSTRACT

Disclosed are methods and compositions for treating biological samples, such as blood, to preserve or enhance the function of the samples.

### FIELD OF THE INVENTION

This invention relates to methods and compositions for enhancing functions of biological compositions that contain red blood cells, platelets or plasma. This invention also relates to improvements in the storage of whole blood and of packed blood cells suitable for transfusion.

### BACKGROUND OF THE INVENTION

Red blood cells (RBCs) that are used for transfusion can be stored for extended periods of time, six weeks or longer. However, stored RBCs suffer “storage lesion”, a series of biochemical and biomechanical changes that lead to hemolysis (breakdown of RBCs) and reduced post-transfusion function and survival. The understanding of the mechanisms involved in the induction of storage lesion is incomplete, but they are related to the decrease of cellular levels of adenosine triphosphate (ATP) and 2,3-diphosphoglycerate (2,3-DPG). Mature erythrocytes depend on ATP to maintain cationic pumps and membrane integrity. The concentration of ATP, after a brief initial rise, progressively declines to between 30 and 40% of its initial level after six weeks of storage. The decline of ATP levels is correlated with lack of phosphorylation of spectrin (key protein regulating RBC membrane mechanical properties), followed by loss of RBC membrane fluidity and integrity and loss of shape and volume of the erythrocyte. In addition, ATP depletion affects fueling ion pumps and channels, the function of RBC enzymes, and the maintenance of phospholipid levels. 2,3-DPG is an intracellular compound that regulates the oxygen transport function of RBCs by modulating the oxygen affinity of hemoglobin. Reduced level of 2,3-DPG results in increased oxygen binding by hemoglobin and decreased oxygen liberation to the tissues. Intracellular 2,3-DPG concentration constantly falls during refrigerated storage of RBC. Usually 2,3-DPG is undetectable after 3-4 weeks of storage. At room temperature 2,3-DPG concentration decreases much faster than at 1-6° C. P50 is a measure of the partial oxygen pressure (pO2, mm Hg) required to achieve 50% oxygen saturation of hemoglobin in RBC. The p50 value of the oxyhemoglobin dissociation curve is highly dependent on intracellular 2,3-DPG level. Although 2,3-DPG depletion is a reversible storage lesion, transfused cells depleted of 2,3-DPG can recover only 50% of their normal level within a 3-8 hour period, possibly not fast enough for compromised or severely ill individual. Thus, the levels of ATP, 2,3-DPG and P50 of red blood cells serve as indicators of the suitability of stored cells for transfusion.

Improved red cell viability has been achieved with storage solutions that are fortified with nutrients and other preservatives, such as phosphate, glucose, and adenine, that are added to maintain the levels of ATP, 2,3-DPG, and P50 and to retard the onset of hemolysis. Numerous additive solutions for RBCs exist either as commercial or research products (see Meryman et al. U.S. Pat. No. 4,585,735; Rock et al. U.S. Pat. No. 4,447,415; Goldstein U.S. Pat. No. 4,427,777; Deniega et al. U.S. Pat. No. 6,527,957; Holme et al. U.S. Pat. No. 5,248,506 the disclosures of which are incorporated herein by reference) that have been devised to reverse the declines in ATP and 2,3-DPG and the morphological changes associated with long-term storage, and thereby enhance the RBC function. One such solution is REJUVESOL™ (Cytosol Laboratories, Braintree, Mass.) that contains 100 mM sodium pyruvate, 100 mM inosine, 5 mM adenine, 70 mM monobasic phosphate and 40 mM dibasic phosphate, at pH 6.7-7.4 (REJUVESOL from hereon). This type of solution, however, is not suitable for transfusion; the components of the solution must be removed from the red blood cells prior to transfusing the cells, typically following 1 hr incubation with REJUVESOL at 37° C., the RBC are washed for 1 hr using 1.5 L of saline and 250 ml of AS-3. Because the red blood cells are subjugated to several washing steps to remove the components there is a risk of contamination associated with this procedure. In addition, transfusion of RBCs poses a risk of pathogen infection in a recipient from blood that has been obtained from donors that are infected with a pathogen, such as hepatitis C virus and/or human immunodeficiency virus.

Because of the aforementioned shortcomings, there is a need to develop methods and solutions that not only maintain high intracellular levels of both ATP and 2,3-DPG, good morphology and low hemolysis but also alleviate the risk of infections (bacterial, viral, fungal, parasitic etc.) by inactivating potential pathogens present in stored RBCs.

### SUMMARY OF THE INVENTION

It now has been discovered that the combination of aziridino compounds and a solution that contains pyruvate, inosine, phosphate and adenine can be used, unexpectedly, to enhance the function of a biological sample, such as a red blood cell solution. Accordingly, improved methods and products for the treatment of biological samples, particularly red blood cells, are provided according to the invention.

According to one aspect of the invention, methods are provided for treatment and enhancement of biological functions of biological samples, particularly red blood cells. These methods include contacting the biological sample with a solution of an aziridino compound in combination with pyruvate, inosine, adenine, and phosphate. In certain embodiments the biological sample includes red blood cells.

Administration of the solution of the aziridino compound in combination with pyruvate, inosine, phosphate and adenine is performed to deliver an effective amount of the solution to the biological sample. Therefore, in some embodiments, the pyruvate is present in the solution at a concentration of about 0.4 to about 40 grams/liter, the inosine is present in the solution at a concentration of about 1 to about 100 grams/liter, the adenine is present in the solution at a concentration of about 0.027 to about 2.7 grams/liter, the phosphate is present as a dibasic phosphate at a concentration of about 0.4 to about 40 grams/liter, the monobasic phosphate is present at a concentration of about 0.16 to about 16 grams/liter, and the aziridino compound is present at a concentration of about 0.01 to about 100 mM. Preferably, the pyruvate is present at a concentration of about 4.4 grams/liter, inosine is present at a concentration of about 10.7 grams/liter, adenine is present at a concentration of about 0.27 grams/liter, and phosphate is present as a dibasic phosphate at a concentration of about 4.0 grams/liter and a monobasic phosphate at a concentration of about 1.6 grams/liter and the aziridino compound is present at a concentration of about 10.7 mM.

In certain embodiments of the foregoing methods, the aziridino compound contains a linear alkyl group. Preferably the aziridino compound has the structure of formula II: 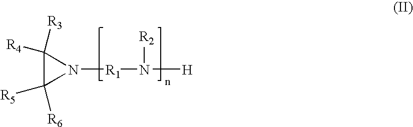
wherein each R₁ is a divalent hydrocarbon moiety containing between two and four carbon atoms, inclusive; each of R₂, R₃, R₄, R₅, and R₆ is, independently, H or a monovalent hydrocarbon moiety containing between one and four carbon atoms, inclusive; and n is an integer between one and ten, inclusive. More preferably, R₂, R₃, R₄, R₅, and R₆ are H.

In other embodiments, the salt of the aziridino compound has the structure of formula 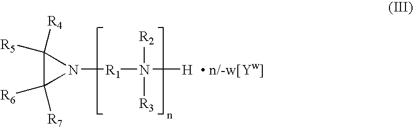
wherein each R₁ is a divalent hydrocarbon moiety containing between two and four carbon atoms, inclusive; each of R₂, R₃, R₄, R₅, R₆, and R₇ is, independently, H or a monovalent hydrocarbon moiety containing between one and four carbon atoms, inclusive; Y is pharmaceutically acceptable counter anion; W is the valency of Y; and n is an integer between one and ten, inclusive. Preferably, R₂, R₃, R₄, R₅, and R₆ are H.

In another embodiment the aziridino compound is an ethyleneimine dimer. In a further embodiment the aziridino compound is an ethyleneimine trimer.

In another aspect of the invention, a method for enhancing the function of a red blood cells is provided by contacting the red blood cells with a solution of an aziridino compound in combination with pyruvate, inosine, phosphate, and adenine. In preferred embodiments, the aziridino compound is an ethyleneimine compound, even more preferably an ethyleneimine dimer, trimer or tetramer.

In another aspect of the invention, methods for enhancing the biological function of red blood cells are provided. These biological functions include, but are not limited to, levels of 2,3-DPG, ATP and p50 in red blood cells wherein the levels of 2,3-DPG, ATP and p50 are increased in the red blood cells treated with the aziridino compound in combination with the pyruvate, inosine, adenine and phosphate in comparison to the levels of 2,3-DPG, ATP and p50 in red blood cells not contacted with the aziridino compound, pyruvate, inosine, adenine and phosphate.

In some embodiments a method for transfusing blood into a subject in provided, wherein the blood sample has been treated by the solution comprising an effective amount of an aziridino compound in combination with pyruvate, inosine, phosphate, and adenine.

In another aspect of the invention a method is provided for enhancing the biological function and selectively inactivating pathogens in a biological sample. The method consists of contacting the biological sample with a solution containing an aziridino compound in combination with pyruvate, inosine, phosphate, and adenine. In some embodiments the biological sample is red blood cells. In preferred embodiments of the foregoing compositions the aziridino compound is an ethyleneimine oligomer, particularly ethyleneimine dimer or ethyleneimine trimer.

In another aspect of the invention, a blood-collecting device is provided that includes a container for receiving blood or a blood fraction, wherein the container contains an aziridino compound in combination with pyruvate, inosine, adenine and phosphate, in an amount effective to enhance the biological function and/or inactivate pathogens in the blood or fraction thereof received into the container. In preferred embodiments of the foregoing compositions the aziridino compound is an ethyleneimine oligomer, particularly ethyleneimine dimer or ethyleneimine trimer.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting. Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

FIG. 1 is a graphical representation of changes in 2,3-DPG levels over time in RBCs treated with various solutions.

FIG. 2 is a graphical representation of changes in P50 levels over time in RBCs treated with various solutions.

FIG. 3 is a graphical representation of changes in ATP levels over time in RBCs treated with various solutions.

FIG. 4 is a graphical representation of changes in PEN110 levels over time in RBCs treated with various solutions.

FIG. 5 is a graphical representation of changes in 2,3-DPG levels over time in RBCs treated with various solutions.

FIG. 6 is a graphical representation of changes in P50 levels over time in RBCs treated with various solutions.

FIG. 7 is a graphical representation of changes in ATP levels over time in RBCs treated with various solutions.

FIG. 8 is a graphical representation of changes in PEN110 levels over time in RBCs treated with various solutions.

FIG. 9 is a graphical representation of changes in 2,3-DPG levels over time in RBCs treated with the INACTINE™ process.

FIG. 10 is a graphical representation of changes in P50 levels over time in RBCs treated with the INACTINE™ process.

FIG. 11 is a graphical representation of changes in ATP levels over time in RBCs treated with the INACTINE™ process.

FIG. 12 is a graphical representation of changes in 2,3-DPG levels over time in RBCs treated with a combination of PEN110 and REJUVESOL solutions.

FIG. 13 is a graphical representation of changes in P50 levels over time in RBCs treated with a combination of PEN110 and REJUVESOL solutions.

FIG. 14 is a graphical representation of changes in ATP levels over time in RBCs treated with a combination of PEN110 and REJUVESOL solutions.

FIG. 15 is a graphical representation of levels of hemolysis over time in RBCs treated with a combination of PEN110 and REJUVESOL solutions.

FIG. 16 is a graphical representation of changes in 2,3-DPG levels over time in RBCs treated with the INACTINE™ process and various REJUVESOL (“Rej”) dilutions (1:20, 1:40, 1:80). The control was conventionally treated RBCs (historical control, N=5).

FIG. 17 is a graphical representation of changes in P50 levels over time in RBCs treated with the INACTINE™ process and various REJUVESOL (“Rej”) dilutions (1:20, 1:40, 1:80).

The control was conventionally treated RBCs (historical control, N=5).

FIG. 18 is a graphical representation of changes in ATP levels over time in RBCs treated with the INACTINE™ process and various REJUVESOL (“Rej”) dilutions (1:20, 1:40, 1:80). The control was conventionally treated RBCs (historical control, N=5).

FIG. 19 is a graphical representation of changes in hemolysis levels over time in RBCs treated with the INACTINE™ process and various REJUVESOL (“Rej”) dilutions (1:20, 1:40, 1:80). The control was conventionally treated RBCs (historical control, N=5).

FIG. 20 is a graphical representation of PEN110 levels in RBC samples treated with the INACTINE™ process and various REJUVESOL (“Rej”) dilutions (1:20, 1:40, 1:80). The control was conventionally treated RBCs (historical control, N=5).

### DETAILED DESCRIPTION OF THE INVENTION

Compositions according to the invention are prepared by combining an aziridino compound and pyruvate, inosine, adenine, and sodium phosphate such that a biological sample, e.g., RBCs, has enhanced biological functions as compared to the same untreated biological sample or when treated with an aziridino compound alone, or when treated with the pyruvate, inosine, adenine and phosphate alone. When the sample includes RBCs, the enhanced biological functions can include one or more of increased 2,3-DPG levels, increased P50 levels, and increased ATP levels.

The aziridino compound preferably is an ethyleneimine oligomer composition known as PEN110. In a particularly preferred embodiment, the aziridino compound is provided by the INACTINE™ process that consists of incubation of the RBCs with 0.1% (v/v) of PEN110 at 23° C. for 24 hours followed by washing of the RBCs by a procedure optimized for the removal of the ethyleneimine oligomer to the level of less than 50 ng/ml.

When the biological sample includes RBCs, pyruvate is preferably present in the treatment solution at a final concentration of about 0.4 to about 40 grams/liter, e.g., about 2.75 to about 6.05 grams/liter, or preferably about 3.85 to about 4.95 grams/liter. A particularly preferred concentration of pyruvate in the solution is about 4.4 grams/liter. Inosine is present in the treatment solution at a concentration of about 1 grams/liter to about 100 grams/liter, e.g., about 6.7 to about 14.74 grams/liter, or preferably about 9.38 to about 12.06 grams/liter. A particularly preferred concentration of inosine in the solution is about 10.7 grams/liter. Adenine is present in the treatment solution at a concentration of about 0.027 to about 2.7 grams/liter, e.g., about 0.17 to about 0.37 grams/liter, or preferably about 0.24 to about 0.31 grams/liter. A particularly preferred concentration of adenine in the solution is about 0.27 grams/liter. Sodium phosphate dibasic is present in the treatment solution at a concentration of about 0.4 to about 40 grams/liter, e.g., about 2.5 to about 5.5 grams/liter, or preferably about 3.5 to about 4.5 grams/liter. A particularly preferred concentration of dibasic sodium phosphate in the solution is about 4.0 grams/liter. Sodium phosphate monobasic is present in the treatment solution at a concentration of about 0.16 to about 16 grams/liter, e.g., about 1.0 to about 2.2 grams/liter or preferably about 1.4 to about 1.8 grams/liter. A particularly preferred concentration of monobasic sodium phosphate in the solution is about 1.6 grams/liter.

A composition of the invention can therefore be prepared by combining an aziridino compound (such as an ethyleneimine oligomer) with one unit of RBCs prepared from 450 ml or 500 ml of whole blood and 50 ml of a solution prepared in sterile water at pH 6.7 to 7.4 and including about 0.22 grams of pyruvate, about 0.536 grams of inosine, about 0.0136 grams of adenine, about 0.2 grams of sodium phosphate dibasic and about 0.08 grams of sodium phosphate monobasic. Pyruvate, inosine, adenine, and sodium phosphate in the compositions of the invention can be provided together in a single solution known as REJUVESOL® blood cell washing solution (Cytosol Laboratories, Braintree, Mass.). REJUVESOL contains 100 mM sodium pyruvate, 100 mM inosine, 5 mM adenine, 70 mM monobasic phosphate and 40 mM dibasic phosphate, at pH 6.7-7.4.

Aziridino compounds useful in the methods and composition of the invention preferably contain a moiety having the formula (I): 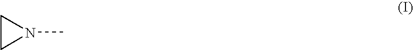
In this three-membered ring, the two carbons are preferably unsubstituted (i.e., they contain hydrogens), but they can be substituted with aliphatic or aromatic hydrocarbon moieties, each containing between one and four carbon atoms, inclusive.

Various aziridino compounds are disclosed in U.S. Pat. No. 6,093,564, and in U.S. application No. 60/379,188, filed on May 6, 2002, entitled Methods and Compositions for the Modification of Nucleic Acids, the entire disclosures of which are incorporated by reference. The use of these compounds for compounds and methods of the invention is provided herein.

In one set of embodiments, the aziridino compound has the formula (II): 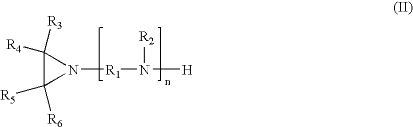
wherein each R₁ is a divalent hydrocarbon moiety containing between two and four carbon atoms, inclusive; each of R₂, R₃, R₄, R₅, and R₆ is, independently, H or a monovalent hydrocarbon moiety containing between one and four carbon atoms, inclusive; and n is an integer between one and ten, inclusive.

In various preferred embodiments, each R₁ contains two or three carbon atoms; each of R₂, R₃, R₄, R₅, and R₆ is H; and n is one, two or three. For example, ethyleneimine tetramer fits formula (II) when R₁ contains two carbon atoms, and each of R₂, R₃, R₄, R₅, and R₆ is H, and n is three. Similarly, ethyleneimine trimer fits formula (II) where R₁ contains two carbon atoms, each of R₂, R₃, R₄, R₅, and R₆ is H, and n is two, and ethyleneimine dimer fits formula (II) when R₁ contains two carbon atoms, and each of R₂, R₃, R₄, R₅, and R₆ is H, and n is one.

In another set of examples, the compound has the formula (III): 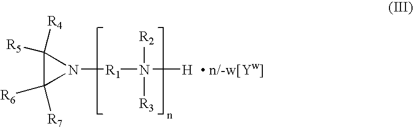
wherein each R₁ is a divalent hydrocarbon moiety containing between two and four carbon atoms, inclusive; each of R₂, R₃, R₄, R₅, R₆, and R₇ is, independently, H or a monovalent hydrocarbon moiety containing between one and four carbon atoms, inclusive; Y is pharmaceutically acceptable counter anion; W is the valency of Y; and n is an integer between one and ten, inclusive.

Aziridino compounds also include open-ring counterparts to the compounds of formula (I). In one example, aziridino compounds useful in the methods of the invention have the formula (IV): 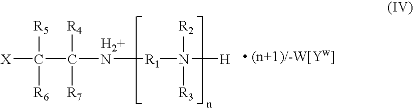
wherein each R₁ is a divalent hydrocarbon moiety containing between two and four carbon atoms, inclusive; each of R₂, R₃, R₄, R₅, R₆, and R₇ is, independently, H or a monovalent hydrocarbon moiety containing between one and four carbon atoms, inclusive; X is Cl or Br; Y is a pharmaceutically acceptable counter anion; W is the valency of Y; and n is an integer between one and ten, inclusive.

In various preferred embodiments of compounds satisfying formula (III) or formula (IV), each R₁ contains two or three carbon atoms; each of R₂, R₃, R₄, R₅, and R₆ is H; and n is one or two. Suitable counter anions include nitrate, sulfate, halide (fluorine, chlorine, bromine, iodine), phosphate, and tosylate ions.

In an additional set of embodiments, the aziridino compound has the formula (V): 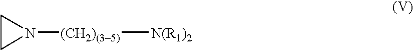
or a salt thereof, wherein each R₁ is, independently, selected from the group consisting of H, C₁₋₄ alkyl, C₂₋₄ alkenyl, phenyl, and benzyl. In particular embodiments, the compound is 1-aziridinepropanamine or 1-aziridinebutanamine (compounds 1 and 2, respectively):

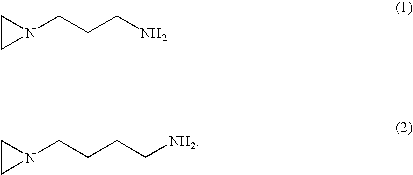

In another additional set of embodiments, the aziridino compound has the formula (VI): 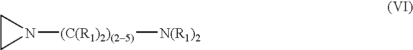
or a salt thereof, wherein each R₁ is, independently, selected from the group consisting of H, C₁₋₄ alkyl, C₂₋₄ alkenyl, phenyl, and benzyl, provided that at least one R₁ is phenyl or benzyl.

Exemplary aziridino compounds that fall within formula (VI) are 3-phenyl-1-aziridinepropanamine, N,N-dibenzyl-1-aziridineethanamine, and N-benzyl-N-ethyl-1-aziridineethanamine, and 2-benzyl-1-aziridineethanamine (compounds 3, 4, 5, and 6, respectively).

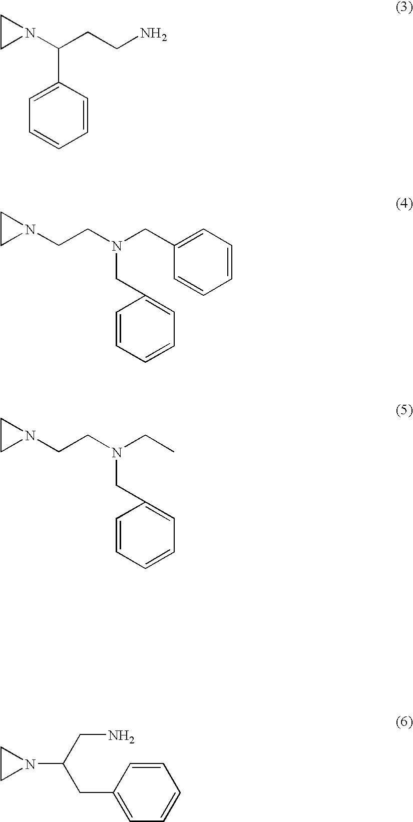

In a further set of embodiments, the aziridino compound has the formula (VII): 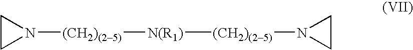
or a salt thereof, wherein R₁ is selected from the group consisting of H, C₁₋₄ alkyl, C₂₋₄ alkenyl, phenyl, and benzyl.

Exemplary compounds that satisfy formula (VII) are 1,1′-[iminobis(dimethylene)]bis aziridine and 1,1′-[iminobis(trimethylene)]bis aziridine (compounds 7 and 8 respectively).

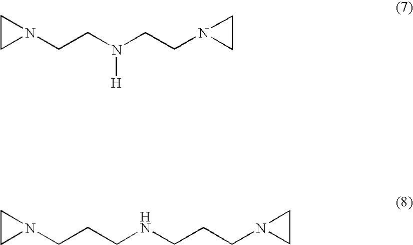

In an additional set of embodiments, the aziridino compound has the formula: 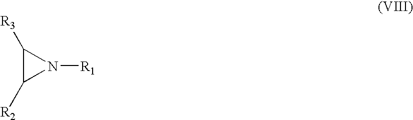
or a salt thereof, wherein R₁ is a C₁₋₄ alkyl and R₂ and R₃ is each, independently, H or a C₁₋₄ alkyl. An exemplary compound of formula (VIII) is:

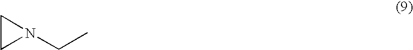

In other embodiments, the aziridino compound is one of the following compounds: 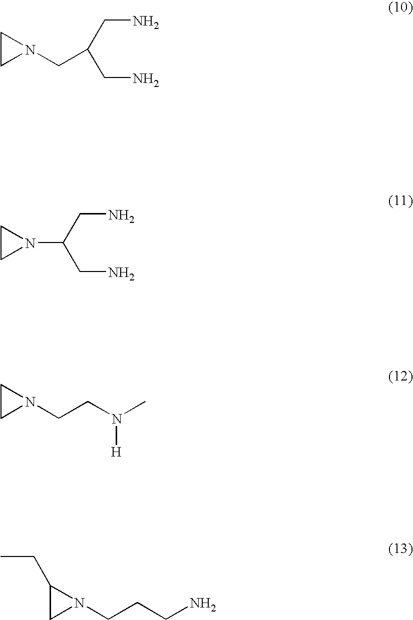
or a salt thereof.

In still another set of embodiments, the aziridino compound has the formula (IX): 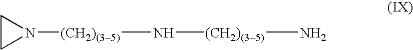
or a salt thereof. An exemplary compound of formula (IX) is:

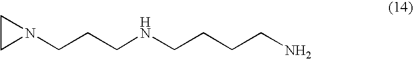

The aziridino ring of the compounds of the invention can be substituted with a structure X—CH₂—CH₂—N—, wherein X is —Cl, —Br, —F, —I, —O—S(═O)₂—CH₃, —O—S(═O)₂—CH₂—C₆H₅, or —O—S(═O)₂—C₆H₄—CH₃. For example, the substituted forms of compounds of formula (V) have the following formula (X):
X—CH₂—CH₂—N—(CH₂)₍₃₋₅₎—N(R₁)₂ (X)
wherein X is —Cl, —Br, —F, —I, —O—S(═O)₂—CH₃, —O—S(═O)₂—CH₂—C₆H₅, or —O—S(═O)₂—C₆H₄—CH₃, each R₁ is, independently, selected from the group consisting of H, C₂₋₄ alkenyl, phenyl, and benzyl.

The aziridino compounds of the present invention are protonated (i.e., positively charged) on one or more nitrogens at physiological pH. For example, protonated compounds of formula (V) (VI), and (VII) have the following respective formulas: 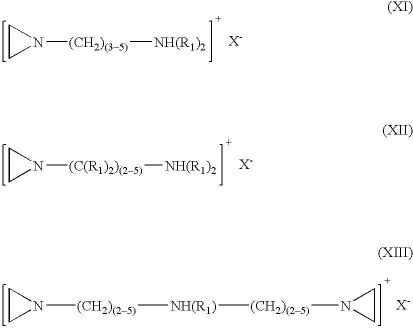
wherein each R₁ is, independently, selected from the group consisting of H, C₂₋₄ alkenyl, phenyl, and benzyl, and X is a pharmaceutically acceptable counter-ion (e.g., sulfate, nitrate, halide, tosylate, phosphate, and the like). For compounds within formula (XII) or (XIII), R₁ can also be C₁₋₄ alkyl. Compounds falling within formula (XII) also have at least one R₁ that is phenyl or benzyl.

These protonated forms of the compounds, described herein, (also referred to as “salts”), and their use in the methods of the invention, are specifically included as being part of the invention.

The compounds useful in the invention described herein also include isomers such as diastereomers and enantiomers, mixtures of isomers, including racemic mixtures, solvates, and polymorphs thereof.

The aziridino compounds are added at a concentration of about 0.0001 M to about 0.015 M, although the concentration can be adjusted higher or lower as needed to provide both inactivation of pathogens and enhancement of biological function.

The aziridino compound and solution containing pyruvate, inosine, adenine, and sodium phosphate can be combined prior to, or after, addition of each ingredient to the biological sample. If desired, the aziridino compound can be removed after treating the sample. Methods for removing include washing (such as centrifugation-based washing) or solid phase based absorbent removal.

As used herein, the term “prevent”, “prevented” or “preventing” and “treat”, “treated” or “treating” when used with respect to the prevention or treatment of an infectious disease refers to a prophylactic treatment which increases the resistance of a biological solution to a microorganism or, in other words, decreases the likelihood that a subject will develop an infectious disease to a microorganism following a transfusion of RBCs treated with the solution containing aziridino compound, pyruvate, inosine, adenine and phosphate.

As used herein, a “subject” shall mean a human, a vertebrate mammal including but not limited to a dog, cat, horse, cow, pig, sheep, goat, or non-human primate, e.g., monkey, or a fowl, e.g., chicken. Included within the scope of the present invention are all animals which are susceptible to infectious diseases and from which are taken biological samples, or to which are administered biological samples.

The aziridino compounds combined with non-aziridino compounds (e.g., pyruvate, inosine, adenine and phosphate) are useful for treating biological samples that will be administered to a subject. These subjects are at risk of developing an infectious disease based on the potential presence of infectious agents in biological samples that are administered to the subjects. For example, a subject at risk of infectious disease is one for whom the exposure to a microorganism or expected exposure to a microorganism is known or suspected. A “subject at risk” of developing an infectious disease as used herein is a subject who has any risk of exposure to a microorganism following transfusion of a biological solution, e.g., someone who is receiving a transfusion of blood or a blood component such as red blood cells.

An “infectious disease” as used herein, refers to a disorder arising from the invasion of a host, superficially, locally, or systemically, by an infectious microorganism. Infectious microorganisms include bacteria, viruses, parasites and fungi.

Infectious bacteria include, but are not limited to, gram negative and gram positive bacteria. Gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pylori, Borrelia burgdorferi, Legionella pneumophilia, Mycobacteria species (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic species), Streptococcus pneumoniae, pathogenic Campylobacter species, Enterococcus species, Haemophilus influenzae, Bacillus antracis, Corynebacterium diphtheriae, Erysipelothrix rhusiopathiae, Clostridium perfringens, Clostridium tetani, Enterobacter aerogenes, Klebsiella pneumoniae, Pasteurella multocida, Bacteroides species, Fusobacterium nucleatum, Streptobacillus moniliformis, Treponema pallidium, Treponema pertenue, Leptospira species, Rickettsia species, and Actinomyces israelli. Additional exemplary bacteria are Mycoplasma, e.g. Mycoplasma pneumoniae, Chlamydophila, e.g. Chlamydophila pneumoniae, Bartonella species, and Tropheryma whippelii.

Specific examples of viruses that have been found in humans include but are not limited to: Retroviridae (e.g. human immunodeficiency viruses, such as HIV-1 (also referred to as HTLV-III, LAV or HTLV-III/LAV, or HIV-III); and other isolates, such as HIV-LP); Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses, including SARS virus); Rhabdoviridae (e.g. vesicular stomatitis viruses, rabies viruses); Filoviridae (e.g. Ebola viruses); Paramyxoviridae (e.g. parainfluenza viruses, mumps virus, measles virus, respiratory syncytial virus); Orthomyxoviridae (e.g. influenza viruses); Bunyaviridae (e.g. Hantaan viruses, bunyaviruses, phleboviruses and Nairo viruses); Arenaviridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxyiridae (variola viruses, vaccinia viruses, pox viruses); and Iridoviridae (e.g. African swine fever virus); and unclassified viruses (e.g., the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

In addition to inactivating viruses that infect human subjects, the invention is also useful for non-human vertebrates.

Infectious virus of both human and non-human vertebrates, include retroviruses, RNA viruses and DNA viruses. This group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-I, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses that are pathogens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picomavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirus (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyavirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus influenza virus (influenza virus type A, many human subtypes); Swine influenza virus, and Avian and Equine Influenza viruses; influenza type B (many human subtypes), and influenza type C (possible separate genus); the family Paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronaviridae, including Infectious Bronchitis Virus (IBV), Mouse Hepatitis virus, Human enteric corona virus, and Feline infectious peritonitis (Feline coronavirus).

Illustrative DNA viruses that infect vertebrate animals include, but are not limited to: the family Poxyiridae, including the genus Orthopoxvirus (Variola major, Variolaminor, Monkeypox, Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A,B,C,D,E and ungrouped); simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, and the genus Parvovirus (Feline panleukopenia virus, bovine parvovirus, canine parvovirus, Aleutian mink disease virus, etc).

Parasites can be classified based on whether they are intracellular or extracellular. An “intracellular parasite” as used herein is a parasite whose entire life cycle is intracellular. Examples of human intracellular parasites include Leishmania, Plasmodium, Trypanosoma cruzi, Toxoplasma gondii, Babesia, and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at lest one obligate intracellular stage in their life cycles. This latter category of parasites includes Trypanosoma rhodesiense and Trypanosoma gambiense, Isospora, Cryptosporidium, Eimeria, Neospora, Sarcocystis, and Schistosoma. An exemplary and non-limiting list of parasites for some aspects of the invention is provided herein.

Blood-borne and/or tissues parasites include Plasmodium, Babesia microti, Babesia divergens, Leishmania tropica, Leishmania, Leishmania braziliensis, Leishmania donovani, Trypanosoma gambiense and Trypanosoma rhodesiense (African sleeping sickness), Trypanosoma cruzi (Chagas' disease), and Toxoplasma gondii.

Typical parasites infecting horses are Gasterophilus; Eimeria leuckarti, Giardia; Tritrichomonas equi; Babesia (RBCs), Theileria equi; Trypanosoma; Klossiella equi; Sarcocystis.

Typical parasites infecting swine include Eimeria bebliecki, Eimeria scabra, Isospora suis, Giardia; Balantidium coli, Entamoeba histolytica; Toxoplasma gondii and Sarcocystis, and Trichinella spiralis.

The major parasites of dairy and beef cattle include Eimeria, Cryptosporidium, Giardia; Toxoplasma gondii; Babesia bovis (RBCs), Babesia bigemina (RBCs), Trypanosoma (plasma), Theileria (RBC); Theileria parva (lymphocytes); Tritrichomonas foetus; and Sarcocystis.

Typical parasites infecting sheep and goats include Eimeria, Cryptosporidium, Giardia; Toxoplasma gondii; Babesia (RBC), Trypanosoma (plasma), Theileria (RBC); and Sarcocystis.

Typical parasitic infections in poultry include coccidiosis caused by Eimeria acervulina, E. necatrix, E. tenella, Isospora and Eimeria truncata; histomoniasis, caused by Histomonas meleagridis and Histomonas gallinarum; trichomoniasis caused by Trichomonas gallinae; and hexamitiasis caused by Hexamita meleagridis. Poultry can also be infected Emeria maxima, Emeria meleagridis, Eimeria adenoeides, Eimeria meleagrimitis, Cryptosporidium, Eimeria brunetti, Emeria adenoeides, Leucocytozoon, Plasmodium, Hemoproteus meleagridis, Toxoplasma gondii and Sarcocystis.

Parasitic infections also pose serious problems in laboratory research settings involving animal colonies. Some examples of laboratory animals intended to be treated, or in which parasite infection is sought to be prevented, by the methods of the invention include mice, rats, rabbits, guinea pigs, nonhuman primates, as well as the aforementioned swine and sheep.

Typical parasites in mice include Leishmania, Plasmodium berghei, Plasmodium yoelii, Giardia muris, Hexamita muris; Toxoplasma gondii; Trypanosoma duttoni (plasma); Klossiella muris; Sarcocystis. Typical parasites in rats include Giardia muris, Hexamita muris; Toxoplasma gondii; Trypanosoma lewisi (plasma); Trichinella spiralis; and Sarcocystis. Typical parasites in rabbits include Eimeria; Toxoplasma gondii; Nosema cuniculi; Eimeria stiedae, and Sarcocystis. Typical parasites of the hamster include Trichomonas; Toxoplasma gondii; Trichinella spiralis; and Sarcocystis. Typical parasites in the guinea pig include Balantidium caviae; Toxoplasma gondii; Klossiella caviae; and Sarcocystis.

Infectious fungi can cause systemic or superficial infections. Primary systemic infection can occur in normal healthy subjects and opportunistic infections, are most frequently found in immuno-compromised subjects. The most common fungal agents causing primary systemic infection include Blastomyces, Coccidioides, and Histoplasma. Common fungi causing opportunistic infection in immuno-compromised or immunosuppressed subjects include, but are not limited to, Candida albicans (an organism which is normally part of the respiratory tract flora), Cryptococcus neoformans (sometimes in normal flora of respiratory tract), and various Aspergillus species.

Other medically relevant microorganisms and the diseases they cause have been described extensively in the literature, e.g., see C. G. A. Thomas, Medical Microbiology, Bailliere Tindall, Great Britain 1983, the entire contents of which is hereby incorporated by reference. Each of the foregoing lists is illustrative, and is not intended to be limiting.

The term “effective amount” of an aziridino compound (optionally combined with other non-aziridino compounds as described herein) refers to the amount necessary or sufficient to realize a desired biologic effect. For example, an effective amount of an aziridino compound and non-aziridino compounds for enhancing the function of a biological sample is that amount necessary to slow the decrease in the levels of, maintain the levels of, or increase the levels of 2,3-DPG, p50 and ATP in biological sample comprised of red blood cells in comparison to a sample not treated with the solution comprising the combination of aziridino compound and non-aziridino compounds (e.g., pyruvate, inosine, adenine and phosphate). The effective amount for any particular application can vary depending on such factors as the particular aziridino compound and/or non-aziridino compounds used.

The aziridino compounds can be used also on the basis of volume/volume amounts. Preferred volume/volume concentrations include from about 0.0001% to about 1.0% vol./vol. Higher concentrations, if necessary for effective treatment, may be achieved by using greater amounts of the aziridino compounds.

For any compound described herein an effective amount can be initially determined from in vitro assays and/or based on known effective amounts for known agents. For instance, the effective amount of aziridino compounds useful for inactivating pathogens and for enhancing biological function can be assessed using standard in vitro assays. These assays can be used to determine an effective amount of the particular aziridino compound.

Effective amounts can also be determined from animal models as will be well known to and routinely performed by one of ordinary skill in the art. Adjusting the dose to achieve maximal efficacy based on the methods described above and other methods as are well-known in the art is well within the capabilities of the ordinarily skilled artisan. Doses of non-aziridino compounds (e.g., pyruvate, inosine, adenine and phosphate) can be adjusted when they are combined with aziridino compounds by routine experimentation, based on the teachings within the specification.

The compositions are mixed with the biological sample for a desired length of time. When the biological sample includes RBCs, a suitable time is one hour at 23° C. to 37° C., although shorter times such as 10 min, 20 min, 30 min, 40 min or 50 min also may be suitable. Longer incubation times include 2 hr, 3 hr, 4 hr, 5 hr, 6 hr, 7 hr, 8 hr, 10 hr, 12 hr, 15 hr, 20 hr, 24 hr, 2 days, 3 days, and so on. Generally, longer incubation times are used with lower incubation temperatures.

A suitable biological sample in certain embodiments includes a blood cell suspension. In some embodiments, the blood cell suspension includes mammalian blood cells. Preferably, the blood cells are obtained from a human, a non-human primate, a dog, a cat, a horse, a cow, a goat, a sheep or a pig. In preferred embodiments, the blood cell suspension includes RBCs and/or platelets and/or leukocytes and/or bone marrow cells.

As noted above, aziridino compound and pyruvate, inosine, adenine, and sodium phosphate can also be used to remove a pathogen from the biological sample. Pathogens whose removal is desired from the biological sample include, e.g., prokaryotic, eukaryotic, viral, and non-viral acellular pathogens. Non-viral acellular pathogens can include, e.g., prions.

The invention also includes biological samples treated by the herein described methods, as well as methods for using the treated biological samples. For example, blood cells treated using the methods and/or compositions described herein can be transfused (either heterologously or autologously) into a subject.

Also provided in accordance with the invention is a kit that includes in one or more vials or containers containing an aziridino compound and non-aziridino compounds such as pyruvate, inosine, adenine, and phosphate, each of which can be provided in a single vial or container, if desired. The kits are optionally provided with instructions for using the compositions in the vials.

The invention additionally provides blood-collection devices comprising a container for receiving blood or a fraction thereof, the container comprising an aziridino compound (such as an ethyleneimine oligomer), and non-aziridino compounds such as pyruvate, inosine, adenine and phosphate in an amount effective to inactivate viruses in blood or a fraction thereof received into the container.

The invention will be further illustrated in the following non-limiting examples.

### EXAMPLES

### Example 1

### Biochemical Functions of RBCs Treated with a Combination of PEN110 and REJUVESOL Solutions

The purpose of this study was to examine the levels of 2,3-DPG, P50, and ATP in RBCs that have been treated with a combination of PEN110 (ethyleneimine oligomer solution) and REJUVESOL™ (100 mM sodium pyruvate, 100 mM inosine, 5 mM adenine, 70 mM sodium phosphate dibasic and 40 mM sodium phosphate monobasic) and then stored at 4° C. One unit of RBCs prepared from 450 ml of whole blood was mixed with PEN110 and/or 50 ml volume of REJUVESOL. Control cells were left untreated. After 24 hours of incubation at 23° C. the cells were washed with the blood cell washing solution and supplemented with AS-3 after the washing step (Valeri et al., Transfusion 40:1341-45, 2000), and the cells were stored at 4° C. The levels of 2,3-DPG, P50, and ATP in the treated cells were examined at various time points. The measured levels of 2,3-DPG, P50, and ATP were compared between four treatments: untreated control, PEN110 only, REJUVESOL only, and PEN110 and REJUVESOL.

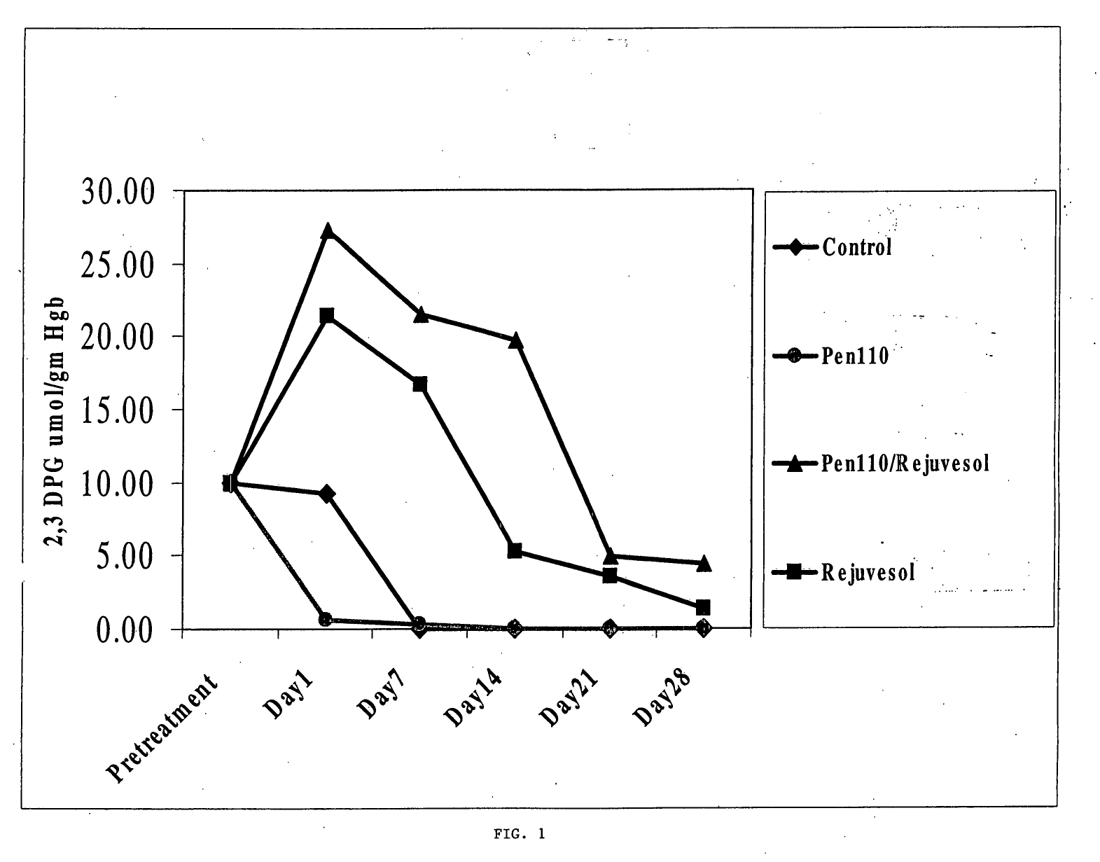

Results of testing for levels of 2,3-DPG are shown in FIG. 1. The levels of 2,3-DPG in treated cells were measured as μmol/gm Hgb. Measurements were taken at Day 1, Day 7, Day 14, and Day 28 following treatment. At all time points examined, cells treated with REJUVESOL (squares) showed elevated levels of 2,3-DPG in comparison to the control (diamonds). The cells that were treated with PEN110 alone (circles) did not show increased levels of 2,3-DPG in comparison to the untreated control at any of the time points examined. Surprisingly, at all time points tested the cells simultaneously treated with the combination of PEN110 and REJUVESOL (triangles) showed higher levels of 2,3-DPG relative to the cells treated with REJUVESOL alone.

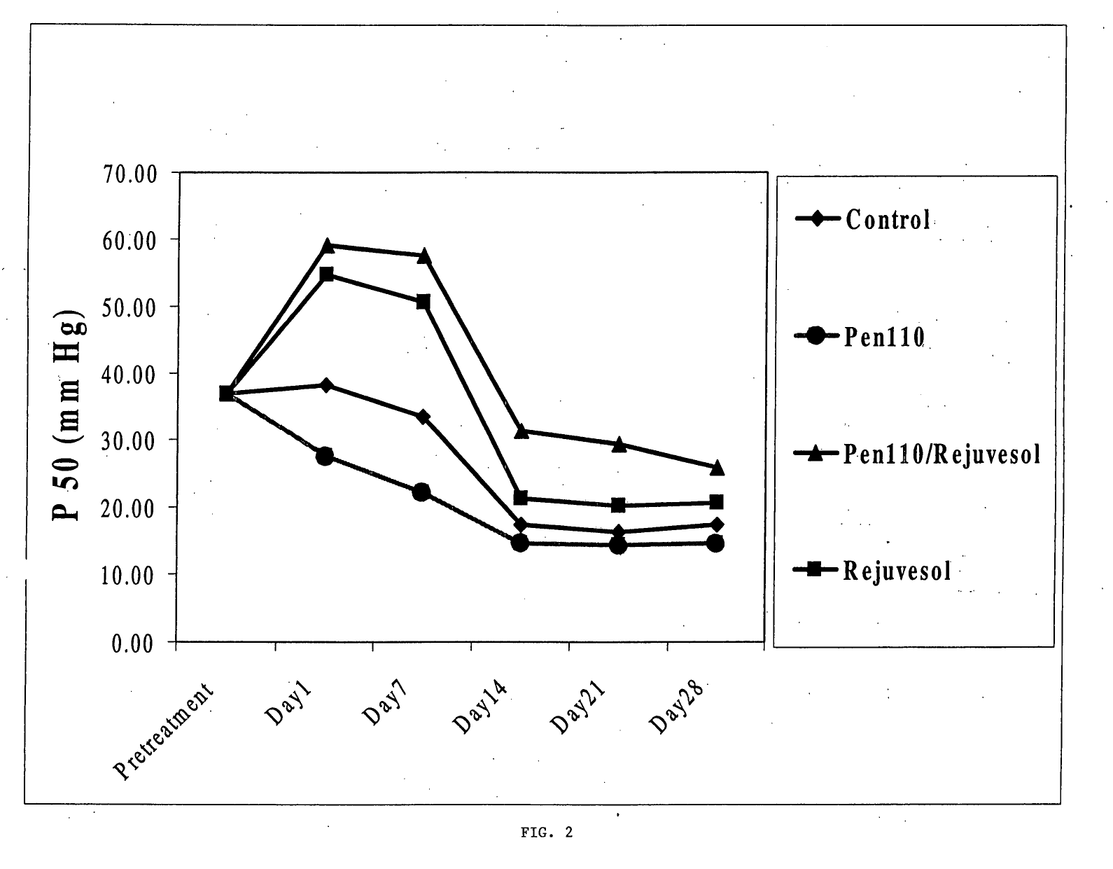

The changes in P50 levels (mm Hg) over time in the treated samples are presented in FIG. 2. Measurements were taken at Day 1, Day 7, Day 14, and Day 28 of storage following treatment. At all time points examined, cells treated with REJUVESOL (squares) showed elevated levels of P50 in comparison to the control (diamonds). The cells that were treated with PEN110 alone (circles) did not show increased levels of P50 in comparison to the control at any of the time points examined. Surprisingly, at all time points tested the cells treated with a combination of PEN110 and REJUVESOL (triangles) showed higher levels of P50 relative to the cells treated with REJUVESOL alone.

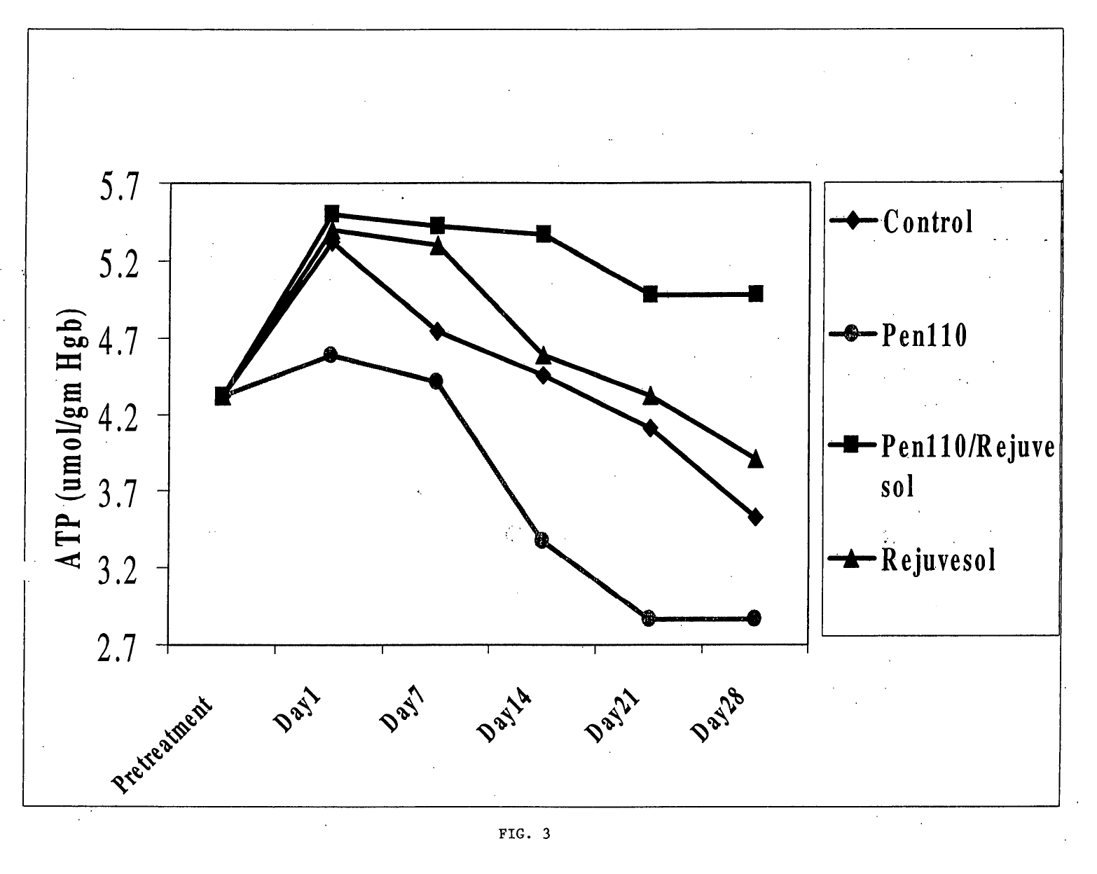

The changes of ATP levels in the samples over time were also measured and are reported in FIG. 3 as μmol/g Hgb. Measurements were taken in cells at Day 1, Day 7, Day 14 and Day 28 of storage following treatment. At all time points examined, cells treated with REJUVESOL (triangles) showed elevated levels of ATP in comparison to the control (diamonds). The cells that were treated with PEN110 alone (circles) did not show increased levels of ATP in comparison to the untreated control at any of the time points examined. Surprisingly, at all time points tested the cells treated with the combination of PEN110 and REJUVESOL (squares) showed higher levels of ATP relative to the cells treated with REJUVESOL alone.

These results demonstrated that RBCs treated with the combination of PEN110 and REJUVESOL showed enhanced biochemical functions as compared to cells treated with PEN110 alone, or cells treated with a REJUVESOL solution alone.

### Example 2

### Effects of REJUVESOL Concentrations on Ethyleneimine Oligomer Removal from PEN110 Treated RBCs

This study was a dose response experiment that compared the effects of various dilutions of REJUVESOL on the removal of ethyleneimine oligomer following PEN110 treatment of a RBC sample. Ethyleneimine oligomer removal from the RBC sample following PEN110 treatment is desirable because of further uses and applications for the treated samples. One unit of RBCs prepared from 450 ml of whole blood was mixed with a 50 ml volume holding 100%, 66%, 40%, 34% or 20% dilutions of REJUVESOL. After 24 hours of incubation at 23° C. the cells were washed with the blood cell washing solution, supplemented with AS-3 after the last step of washing (Valeri et al., Transfusion 40:1341-45, 2000) and stored at 4° C.

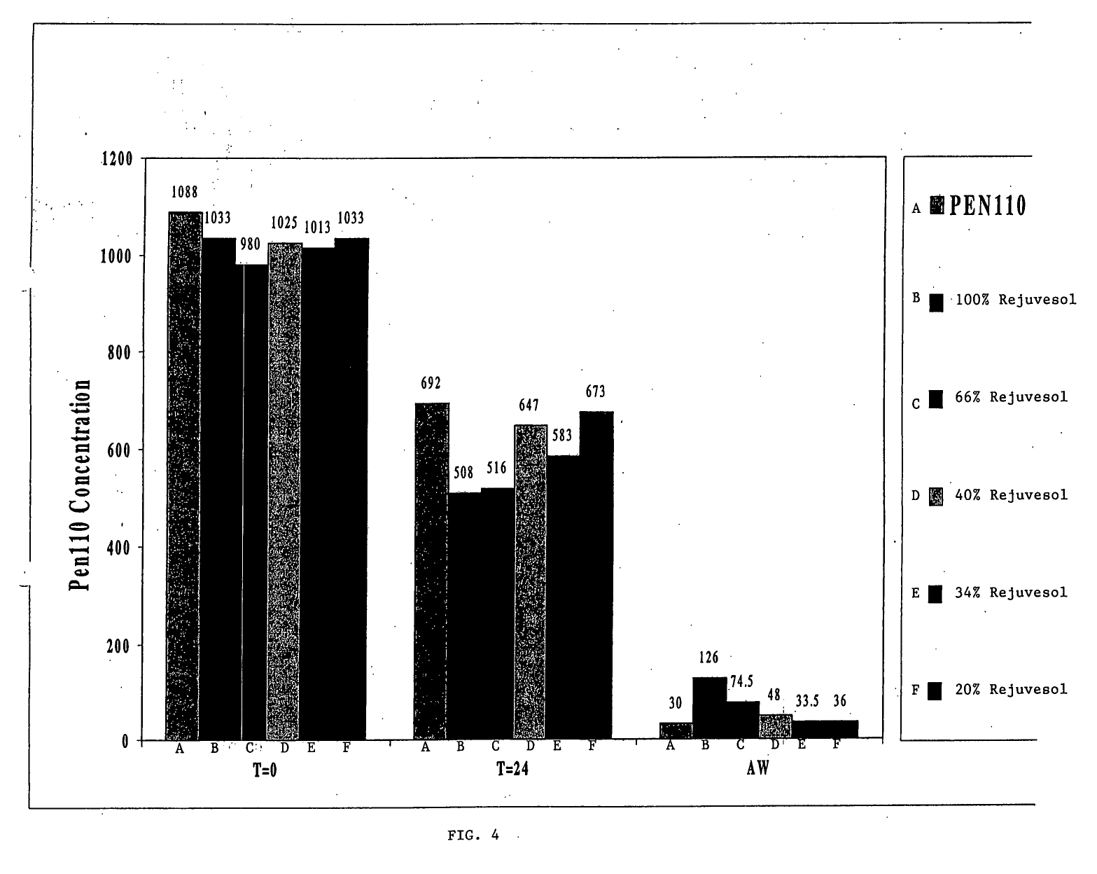

The levels of ethyleneimine oligomer were examined immediately after adding treatment components to the blood (T=0), after 24 hrs of treatment (T=24) and after automatic cell washing (AW). The results are presented in FIG. 4. The control in this experiment were samples treated with PEN110 only.

The levels of ethyleneimine oligomer for all samples at T=0 are shown in the left-hand set of bars in FIG. 4. The concentration of ethyleneimine oligomer at T=0 for the sample treated only with PEN110 was determined to be 1088 μg/ml. The levels of ethyleneimine oligomer at T=0 were similar for all of the REJUVESOL dilutions tested. They ranged from 980 to 1033 μg/ml and were comparable to the PEN110 treated control.

The levels of ethyleneimine oligomer for all samples at T=24 are shown in the central set of bars in FIG. 4. The concentration of ethyleneimine oligomer at T=24 for the control (treated with PEN110 only) was determined to be 692 μg/ml. For the samples treated with the various dilutions of REJUVESOL, the levels of ethyleneimine oligomer at T=24 were similar for all dilutions tested. They ranged from 508 to 673 μg/ml and they were comparable to the PEN110 treated control, the greatest difference being a 1.3-fold decrease in the ethyleneimine oligomer level between the control (sample treated with PEN110 only) and the sample treated with 100% REJUVESOL solution.

The levels of ethyleneimine oligomer for all samples were determined following automated washing (AW) and are shown right hand set of bars in FIG. 4. The AW was carried out by a Haemonetics 215 cell washer (Haemonetics Corporation, Braintree, Mass.) and PBS as a washing solution according to the manufacturer's instructions. The concentration of ethyleneimine oligomer at AW for the control (treated with PEN110 only) was determined to be 30 μg/ml. At this time point, there was a significant difference observed in the levels of ethyleneimine oligomer for the various dilutions of REJUVESOL. The samples treated with 40%, 34% and 20% dilutions of REJUVESOL showed comparable levels of ethyleneimine oligomer as the control sample: 48, 33.5, and 36 μg/ml, respectively. In comparison, the samples that were treated with 100% and 66% dilutions of REJUVESOL showed 4.6-fold and 2.5-fold higher levels of ethyleneimine oligomer concentration, 126 and 74.5 μg/ml, respectively.

These results suggest that at T=0 and T=24 there are small differences in ethyleneimine oligomer removal following sample treatment with 20-100% dilutions of REJUVESOL. In contrast, following automatic washing there were differences in the removal of ethyleneimine oligomer from the treated samples between the various REJUVESOL dilutions.

### Example 3

### Effects of REJUVESOL Concentration on the Biochemical Functions of PEN110 Treated RBCs

The purpose of this experiment was to determine what dilutions of REJUVESOL enhance the biochemical properties of stored RBCs, without the negatively effecting the removal of the ethyleneimine oligomer from the sample. It was determined in the previous example that following automated washing only 40%, 34% and 20% solutions of REJUVESOL did not affect the removal of ethyleneimine oligomer from the PEN110 treated sample.

One unit of RBCs prepared from 450 ml of whole blood was mixed with a 50 ml volume holding 100%, 66%, 40%, 34% or 20% of REJUVESOL. After 24 hour incubation at 23° C. with PEN110 the cells were washed with RBC washing solution and supplemented with AS-3 after the last step of washing (Valeri et al., Transfusion 40:1341-45, 2000) and stored at 4° C.

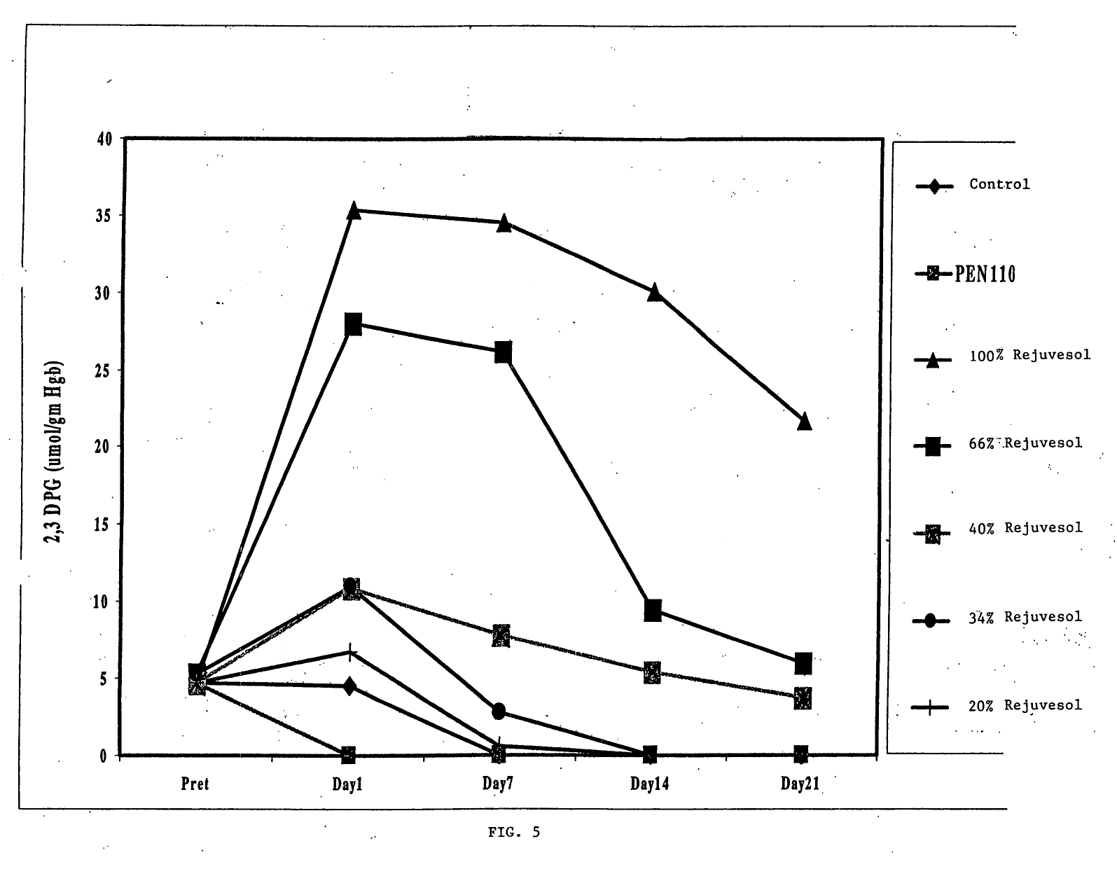

The results of testing for levels of 2,3-DPG are shown in FIG. 5. The levels of 2,3-DPG in treated cells over time were measured as μmol/gm Hgb. Measurements were taken at Day 1, Day 7, Day 14, and Day 21 following treatment. At all dilutions examined, cells treated with REJUVESOL showed higher levels of 2,3-DPG relative to the untreated cells (diamonds), or cells treated with PEN110 (gray squares). The 2,3-DPG levels showed a dose response to REJUVESOL: the highest enhancement for 2,3-DPG levels was observed for the 100% REJUVESOL; followed by the 66% REJUVESOL dilution; followed by the 40% REJUVESOL dilution and so on. The 40% (X squares), 34% (circles), and 20% (crosshairs) REJUVESOL dilutions had shown no effect on ethyleneimine oligomer removal following automated washing (Example 2), and these dilutions all showed enhanced levels of 2,3-DPG at the Day 1 time point. However, at the Day 14 time point only the 40% REJUVESOL dilution showed enhanced levels of 2,3-DPG in comparison to the control. The 34% and the 20% REJUVESOL dilutions showed no detectable levels of 2,3-DPG at the Day 14 or the Day 21 time points. These results showed that treatment of RBCs samples with REJUVESOL dilutions benefits both the 2,3-DPG levels of the RBCs and the ethyleneimine oligomer removal.

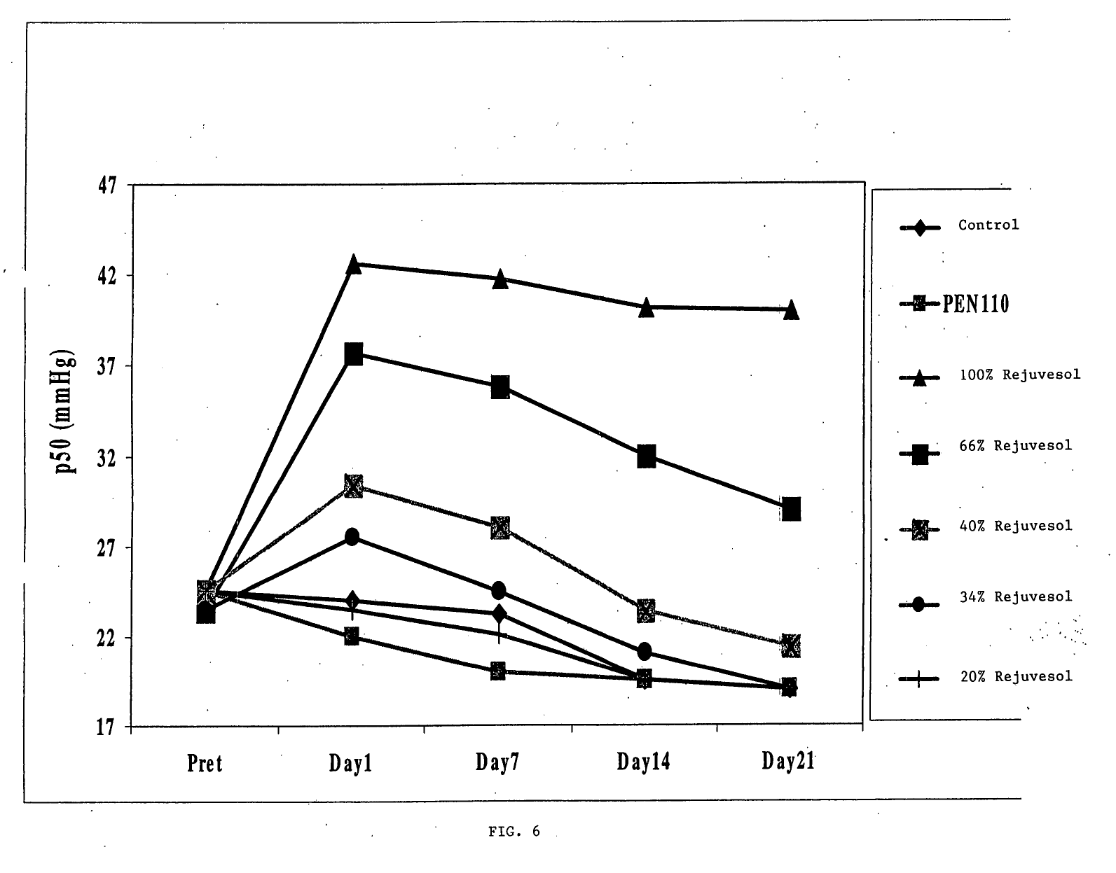

The P50 levels (mm Hg) of RBCs treated with REJUVESOL dilutions are presented in FIG. 6. Measurements were taken at Day 1, Day 7, Day 14, and Day 21 following treatment. At all time points examined, cells treated with the REJUVESOL dilutions showed a concentration dependent dose response and elevated levels of P50 in comparison to the untreated control (diamonds); except for the 20% REJUVESOL dilution (crosshairs), which showed no enhanced effect on the P50 level in comparison to the untreated control. The cells that were treated with PEN110 alone (gray squares) did not show enhanced P50 levels in comparison to the control (diamonds) at any of the time points examined. The 40% REJUVESOL dilution (X squares) showed elevated P50 levels relative to the control cells for all of the time points tested. These results showed that treatment of a RBCs sample with REJUVESOL dilutions can enhance the levels of both, P50 and 2,3-DPG of treated RBCs.

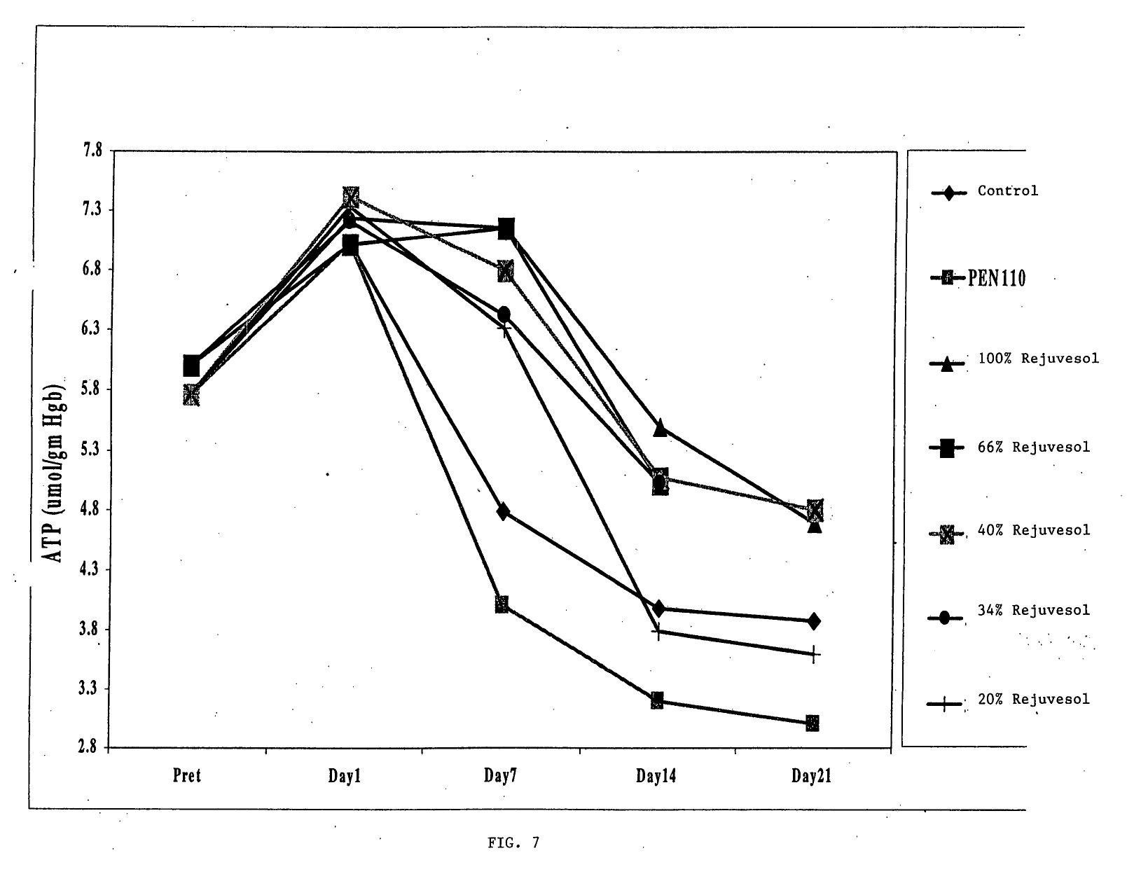

The levels of ATP in the RBC samples treated with REJUVESOL dilutions are reported in FIG. 7 as μmol/gm Hgb. Measurements were taken at Day 1, Day 7, Day 14, and Day 21 of storage following treatment. For all REJUVESOL dilutions the treated cells showed higher ATP levels relative to the untreated control cells (diamonds). The cells that were treated with PEN 10 alone (gray squares) did not show increased levels of ATP in comparison to the control (diamonds) at any of the time points examined. There was no concentration dependent dose response observed however, and at all time points where measurements were taken the ATP levels for the 40% REJUVESOL dilution treatment (X squares) were comparable to the ATP levels measured for the 66% (black squares) or the 100% (triangles) REJUVESOL dilution.

### Example 4

### Effects of pH and REJUVESOL Concentration on Ethyleneimine Oligomer Removal Following PEN110 Treatment of Red Blood Cell Samples

This study was performed to compare the effects of pH on ethyleneimine oligomer removal from RBC samples treated with PEN110 and a 1:2.5 REJUVESOL dilution (40%). One unit of RBCs prepared from 450 ml of whole blood was mixed with a 50 ml volume holding a 1:2.5 REJUVESOL dilution (the approximate concentration of the solution was 40 mM sodium pyruvate, 40 mM inosine, 2 mM adenine, 28 mM sodium phosphate dibasic and 16 mM sodium phosphate monobasic). After 24 hour of incubation with PEN110 at 23° C. the cells were washed with the blood cell washing solution and supplemented with AS-3 after the last step of washing (Valeri et al., Transfusion 40:1341-45, 2000), and stored at 4° C.

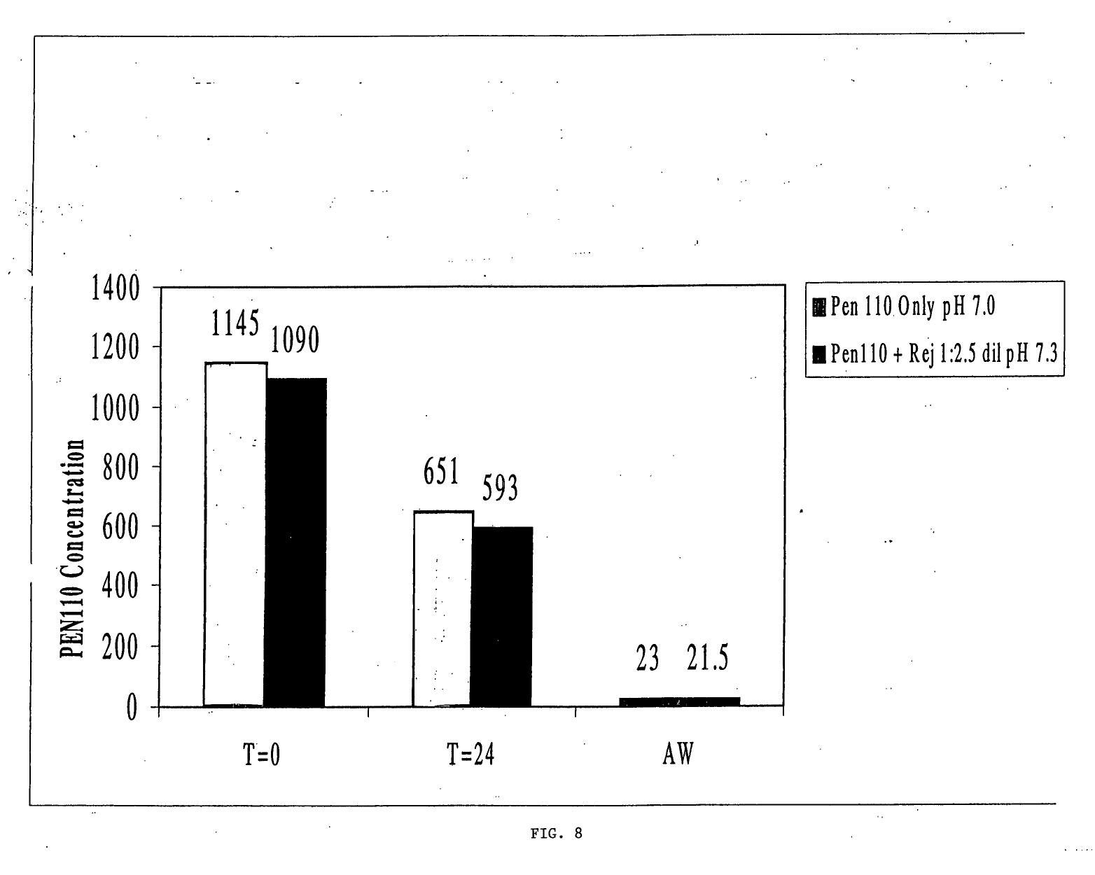

The levels of ethyleneimine oligomer were examined immediately after treatment (T=0), 24 hrs after treatment (T=24), and after automatic washing (AW) and compared to levels of ethyleneimine oligomer in samples that were treated with a 1:2.5 REJUVESOL dilution in addition to PEN110. The results are presented in FIG. 8. The ethyleneimine oligomer levels in samples treated only with PEN110 at pH 7.0 are indicated with a white (clear) bar. Levels of ethyleneimine oligomer in samples treated at pH 7.3 with PEN110 and a 1:2.5 REJUVESOL dilution are indicated as black bars.

The levels of ethyleneimine oligomer for all samples at T=0 are shown in the left-hand set of bars in FIG. 8. The concentration of ethyleneimine oligomer at T=0 for the sample treated only with the PEN110 was determined to be 1145 μg/ml. The levels of ethyleneimine oligomer at T=0 for the sample that was treated with both PEN110 and the REJUVESOL dilution were determined to be 1090 μg/ml, a value comparable to the PEN110 treated control. The levels of ethyleneimine oligomer for the samples at T=24 are shown in the central panel in FIG. 8. The concentration of ethyleneimine oligomer at T=24 for the control (treated with PEN110 only) was determined to be 651 μg/ml. For the sample treated with PEN110 and the REJUVESOL dilution the levels of ethyleneimine oligomer at T=24 were similar, and actually slightly lower than the control, 593 μg/ml.

The levels of ethyleneimine oligomer for the samples following automated washing (AW) using a Haemonetics 215 cell washer and PBS as a washing solution, are shown in the right-hand set of bars. The concentration of ethyleneimine oligomer at AW for the control (treated with PEN110 only) was determined to be 23 μg/ml. At this time point, there was no significant difference observed in the levels of ethyleneimine oligomer for the two samples tested. The samples treated with 1:2.5 REJUVESOL dilution showed comparable levels of ethyleneimine oligomer as the control sample, about 21.5 μg/ml.

These results suggest that under pH of 7.3 there are no significant negative effects on ethyleneimine oligomer removal following sample treatment with PEN110 and 1:2.5 REJUVESOL dilution, following automated washing.

Taken together with the other examples, these results demonstrate that it is possible to achieve better enhancement of function of a RBCs sample by treatment with a solution containing PEN110 (ethyleneimine oligomer) and REJUVESOL (pyruvate, inosine, adenine and phosphate) in comparison to treatment with just ethyleneimine oligomer or just pyruvate, inosine, adenine and phosphate mixture. Furthermore, it is possible to achieve enhancement of the function of RBC sample treated with PEN110 and REJUVESOL without negatively affecting the removal of ethyleneimine oligomer from the sample.

### Example 5

### Effect of INACTINE™ Process on the Biochemical Properties of Treated and Stored RBC

The purpose of this study was to examine the levels of 2,3-DPG, P50 and ATP in RBCs that have been treated with the INACTINE™ process. For RBC concentrates, the INACTINE™ process consists of incubation of the RBCs with 0.1% (v/v) of PEN 110 (ethyleneimine oligomer) at 23° C. for 24 hours followed by washing of the RBCs by a procedure optimized for the removal of the ethyleneimine oligomer to a level of less than 50 ng/ml.

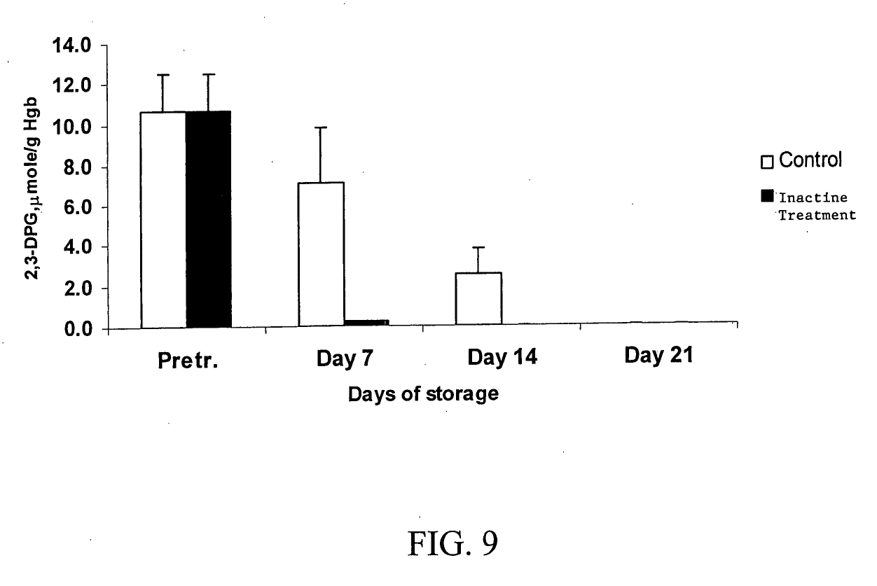

The levels of 2,3-DPG in treated samples were measured as μmol/g Hgb and are shown in FIG. 9 as a summary of six independent experiments. Measurements were taken at Day 1, Day 7, Day 14, and Day 21 following treatment. In the pretreated cells, the levels of 2,3-DPG were about 10 μmol/g Hgb. By Day 7 the levels of 2,3-DPG dramtically decresed in both samples; they were measured at about 7 μmol/g Hgb in the control samples; and about 0.1 μmol/g Hgb in the INACTINE™ treated sample. At Day 14, the levels of 2,3-DPG in the samples continued to decrease, they were at about 3 μmol/g Hgb in the control sample and they were undetectable in the INACTINE™ treated sample. At Day 21 there were no detectable levels of 2,3-DPG in either sample.

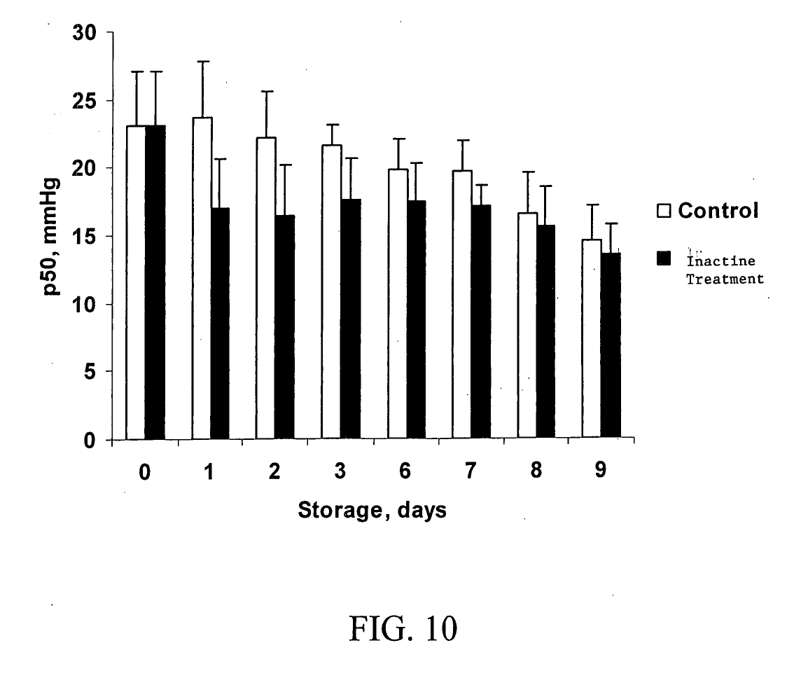

The changes in P50 levels (mm Hg) were determined for up to 9 days of storage for untreated control and INACTINE™ treated samples of RBC, and are shown in FIG. 10 as a summary of six independent experiments. Initially (at Day 0), the P50 levels were comparable between the control and the INACTINE™ treated sample and measured to be about 22 mmHg. At Day 1, the levels of P50 were slightly increased for the control sample-about 22.5 mm Hg, while the P50 levels in the INACTINE™ treated sample decreased to about 16 mmHg. At Day 2, the P50 levels of were slightly decreased for the control sample to about 21 mm Hg, while the P50 levels in the INACTINE™ treated sample slightly decreased in comparison to Day 1 to about 15.5 mmHg. At Day 3, the P50 levels were further slightly decreased for the control sample to about 20.5 mm Hg, while the P50 levels in the INACTINE™ treated sample slightly increased in comparison to Day 2, to about 17.5 mmHg. From Day 3 through Day 9, the levels of p50 in both samples followed a decreasing trend, and ultimately at Day 9 the P50 levels in the control sample decreased to about 14 mmHg and the P50 levels in the INACTINE™ treated sample decreased to about 13 mmHg.

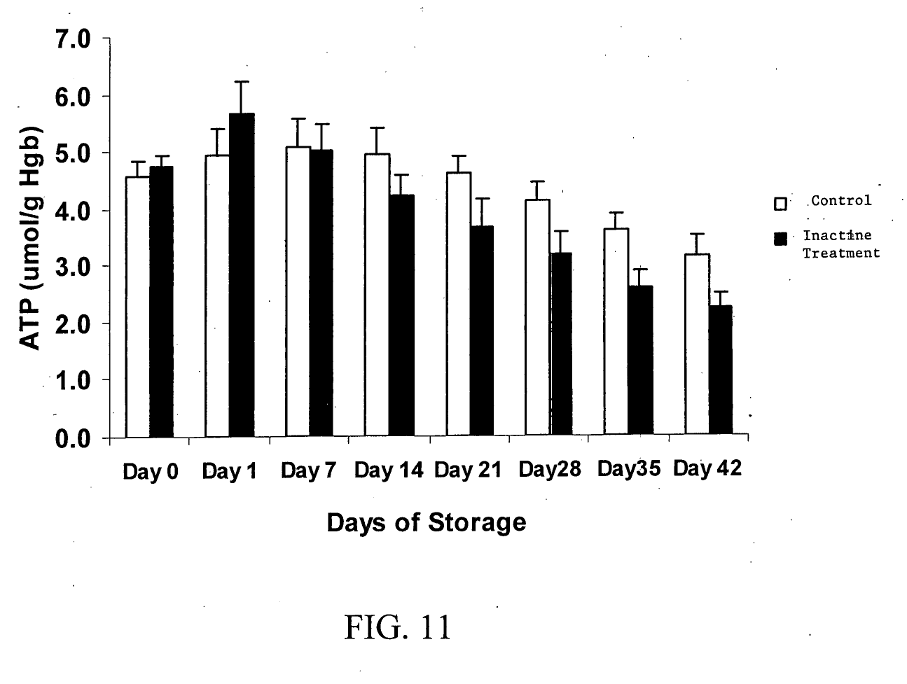

The cellular levels of ATP were determined for the untreated control and INACTINE™ treated samples of RBC for up to 42 days of storage and are shown in FIG. 11 as a summary of six independent experiments. Initially (at Day 0), the levels of ATP were comparable between the control and the INACTINE™ treated sample and measured to be about 4.5 μmol/g Hgb. At Day 1, the levels of ATP were slightly increased for both samples, for the control sample to about 4.9 μmol/g Hgb, and for the INACTINE™ treated sample to about 5.5 μmol/g Hgb. At Day 7, the levels of ATP were slightly decreased for both samples in comparison to Day 1, to about 5.0 μmol/g Hgb. At Day 14, the levels of ATP were slightly decreased for both samples in comparison to Day 7, for the control sample to about 4.9 μmol/g Hgb, and for the INACTINE™ treated sample to about 4.2 μmol/g Hgb. At Day 21, the levels of ATP continued to decrease for both samples, for the control sample to about 4.6 μmol/g Hgb, and for the INACTINE™ treated sample to about 3.8 μmol/g Hgb. From Day 21 though Day 42 the ATP levels followed a decreasing trend. At Day 42, the levels of ATP decreased to about 3.1 μmol/g Hgb for the control sample and 2.1 μmol/g Hgb for the INACTINE™ treated sample.

Taken together, these results demonstrate that RBCs treated with the INACTINE™ process show decreased, but also comparable overall biochemical functions as compared to RBC that were not treated with the INACTINE™ process.

### Example 6

### Rejuvenation of INACTINE™ Treated RBCs by Simultaneous Treatment with PEN110 and REJUVESOL

The purpose of this study was to compare the levels of 2,3-DPG, P50, ATP and hemolysis for RBC samples that were simultaneously treated with PEN110 and REJUVESOL (20 ml, 1:20 dilution) as compared to untreated control samples. The samples were first treated by the INACTINE™ process; in brief, following a 24 hour simultaneous incubation with the PEN110 and REJUVESOL at 23° C., the RBCs were washed with the blood cell washing solution, supplemented with AS-3 after the last step of washing (Valeri et al., Transfusion 40:1341-45, 2000), and stored at 4° C. The untreated control was taken through all the same steps in the procedure. Following treatment, the levels of 2,3-DPG, P50, ATP and hemolysis were determined at various time points for up to 6 weeks of storage.

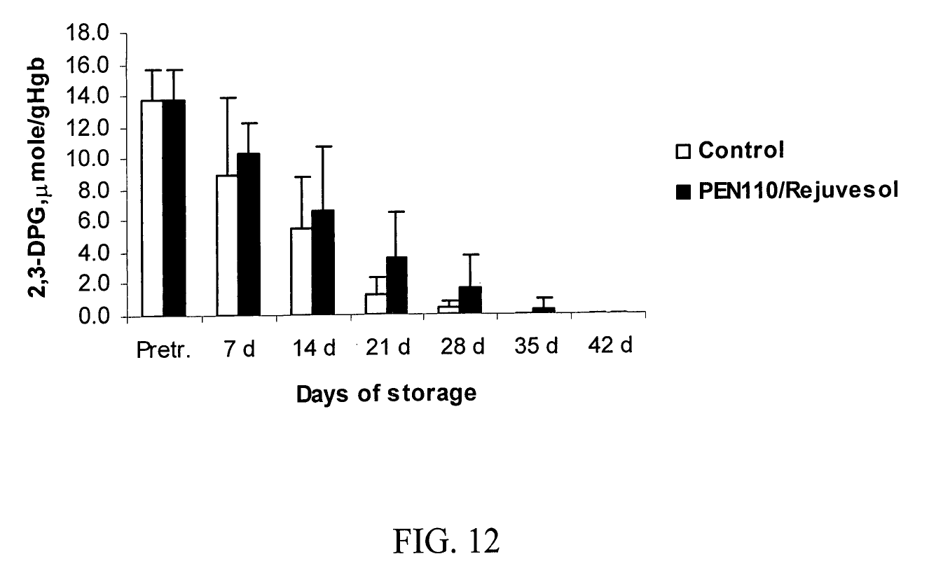

The levels of 2,3-DPG were measured as μmol/gm Hgb for three independent experiments. The RBCs simultaneously treated with PEN110 and REJUVESOL were compared to the control sample. As illustrated in FIG. 12, the pretreated cells showed equivalent amounts of 2,3-DPG of about 13 μmol/g Hgb. During storage from 7 to 42 days, the levels of 2,3-DPG in the control sample significantly declined to about 9 μmol/g Hgb at Day 7, 6.0 μmol/g Hgb at Day 14, 2 μmol/g Hgb at Day 21, and to about 0.5 μmol/g Hgb at Day 28. At Day 35 and at Day 42, there were no detectable levels of 2,3-DPG in the control sample. In comparison, the levels of 2,3-DPG in the sample simultaneously treated with PEN110 and REJUVESOL were higher than the untreated control for all time points tested. At Day 7, the levels of 2,3-DPG were about 10 μmol/g Hgb, comapred to 9 μmol/g Hgb in the control sample. At Day 14, the levels of 2,3-DPG were at about 7 μmol/g Hgb compared to 6.0 μmol/g Hgb in the control sample. At Day 21, the levels of 2,3-DPG were about 4 μmol/g Hgb for the treated sample, again higher than the control sample where the levels were 2 μmol/g Hgb. This trend continued for the remainder of time points examined, at Day 28, the levels of 2,3-DPG in the treated sample were 3 μmol/g Hgb and 0.5 μmol/g Hgb in the control. At Day 35, the levels of 2,3-DPG were about 0.2 to 0.5 μmol/g Hgb in the treated sample and there was no detectible 2,3-DPG in the control. At Day 42, there were no detectable levels of 2,3-DPG for either the control or the treated sample.

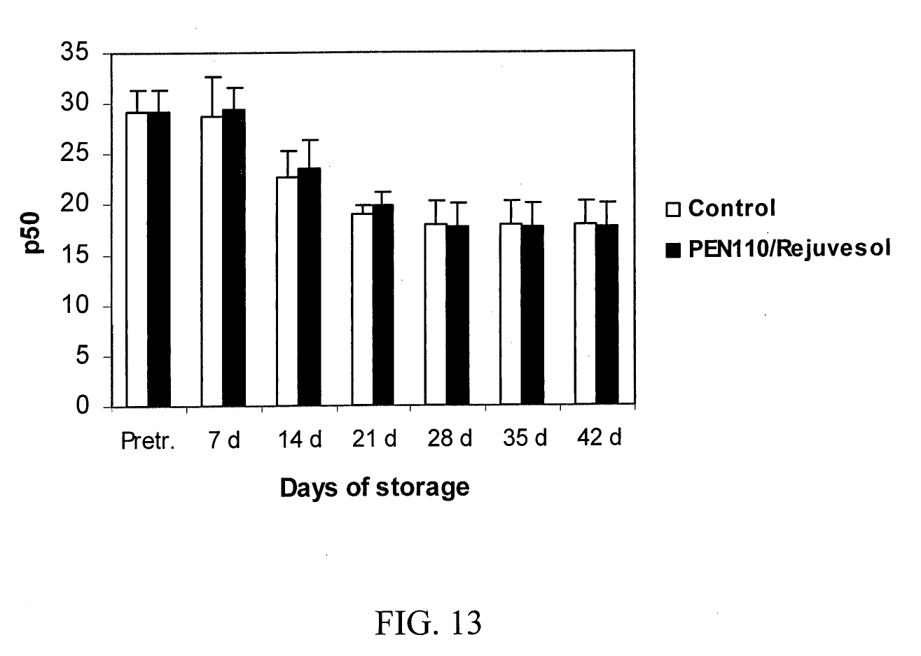

The P50 levels were determined in both samples, the untreated control and the sample simultaneously treated with PEN110 and REJUVESOL, for up to 42 days of storage for three independent experiments and are shown in FIG. 13. Overall, the levels of P50 between the control and the untreated samples were more comparable than the 2,3-DPG levels. Before treatment the P50 levels were determined to be about 28 mm Hg. At Day 7, the P50 levels were determined to be 28 mm Hg and 29 mm Hg for the control and the treated sample respectively. At Day 14, the P50 levels for the samples decreased in comaprison to the pretreatment values, to 22 mm Hg and 23 mm Hg for the control and the treated samples respectively. At Day 21, the P50 levels in both samples continued to desrease to 18 mm Hg and 19 mm Hg, for the untreated and treated sample, respectively. The trend of descreasing P50 values for both samples continued up to the Day 28 measurement, where the P50 values leveled off at about 17 mm Hg for both samples. The P50 values remained at this level through the end of the experiment and were identical at Day 35 and Day 42.

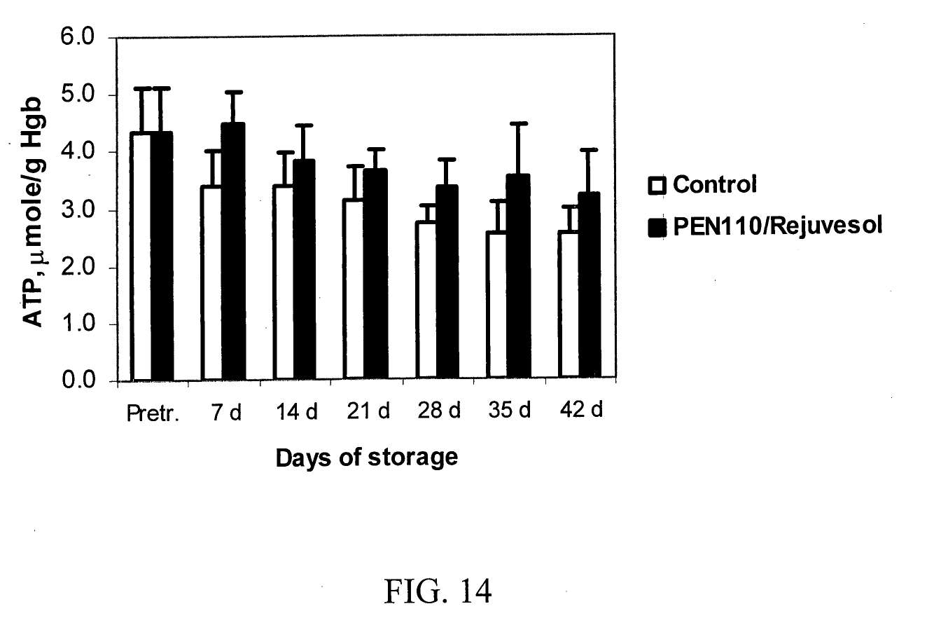

The cellular levels of ATP were compared for both the untreated control sample and the sample simultaneously treated with PEN110 and REJUVESOL, for up to 42 days of storage and are shown in FIG. 14. In the pretreated samples, the levels of ATP were about 4.2 μmol/g Hgb. The ATP levels in the control sample steadily declined during storage from about 3.2 μmol/g Hgb at Day 7, to about 2.9 μmol/g Hgb at Day 42. The levels of ATP in the sample simultaneously treated with PEN110 and REJUVESOL were higher at all time points tested in comparison to the untreated control, and it ranged from 4.3 μmol/g Hgb at Day 7, to about 3.2 μmol/g Hgb at Day 42.

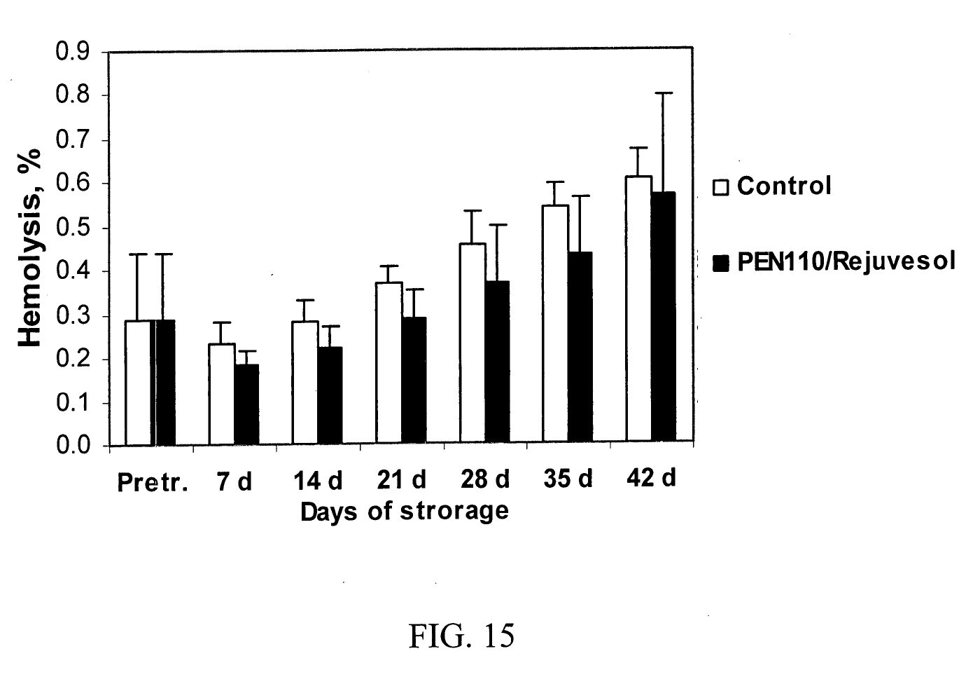

In addition to the 2,3-DPG, P50 and ATP measurements, the percentage of hemolysis was determined in both the control and the sample simultaneously treated with PEN110 and REJUVESOL for up to 42 Days of storage. The results of three independent experiments are shown in FIG. 15. The pretreated samples showed about 30% level of hemolysis. At Day 7, the hemolysis level decreased for both samples, down to 22% and 19% for the control and the treated sample respectively. At Day 14, the hemolysis levels for both samples slightly increased, to about 29% and 22% for the control and the treated sample respectively. At Day 21 the hemolysis levels for both samples continued to increase to 38% and 30% for the control and the treated samples, respectively. This trend continued throughout the storage period, at Day 28, Day 35 and Day 42 the hemolysis levels continued to increase to 60% and 58% for the control and the treated samples respectively. It is noteworthy that for all of the time points tested, the hemolysis levels for the samples simultaneously treated with PEN110 and REJUVESOL were lower than the untreated control.

Taken together, the above experiments show that the simultaneous addition of PEN 10 and REJUVESOL (20 ml, 1:20 dilution), restored the normal physiological levels of 2,3-DPG, P50 and ATP in INACTINE™ treated RBCs.

### Example 7

### Rejuvenation of INACTINE™ Treated RBCs During the Cell Wash

The purpose of this study was to determine if REJUVESOL rejuvenation during the cell wash step of the INACTINE™ process would result in restored and enhanced biochemical function of the tretaed RBCs. The experimental design was as follows: a pool of RBCs prepared from whole blood was treated by PEN110 at 23° C. for 24 hours, followed by a washing step. At this point, the samples were washed either with saline solution or one of three REJUVESOL dilutions: 1:80, 1:40 or 1:20. Following the wash step, all samples were tested for levels of 2,3-DPG, P50, ATP and hemolysis during 6 weeks of storage. It should be noted that the pH of the saline solution fortified with REJUVESOL was dependent on the REJUVESOL concentration. For instance, the saline solution had a pH of 5.74, the 1:80 REJUVESOL dilution had a pH of 7.08, the 1:40 REJUVESOL dilution had a pH of 7.07, and 1:20 REJUVESOL dilution had a pH of 7.11. In addition to the pH, the different REJUVESOL dilutions also differed in osmolarity. The osmolarity of the saline solution was determined to be 292 mOsm, while the osmolarity of the REJUVESOL dilutions of 1:80, 1:40, and 1:20 was determined to be 296 mOsm, 298 mOsm and 302 mOsm, respectively.

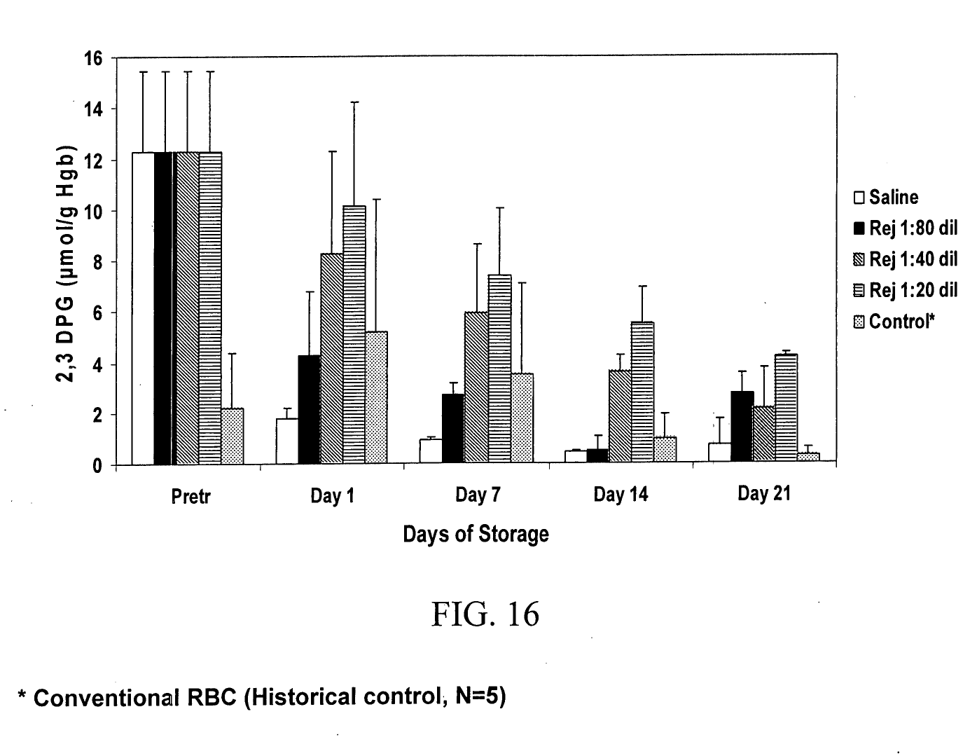

The cellular levels of 2,3-DPG were determined in all four samples following INACTINE™ treatment (saline wash, 1:80 REJUVESOL dilution wash, 1:40 REJUVESOL dilution wash, and 1:20 REJUVESOL dilution wash), and compared to a historical control of conventional RBCs over a period of 21 days of storage. The results of three independent experiments are shown in FIG. 16 (for the historical control the results represent five independent experiments). Before any treatment, the 2,3-DPG level in the various wash samples was measured to be approximately 12 μmol/g Hgb, while the 2,3-DPG level in the historical control was considerably lower, 2 μmol/g Hgb. At Day 1 of storage, the 2,3-DPG levels in all the four samples declined ranging from about 10 μmol/g Hgb for the 1:20 REJUVESOL dilution wash, about 10 μmol/g Hgb for the 1:40 REJUVESOL dilution wash, about 4 μmol/g Hgb 1:80 REJUVESOL dilution wash, to about 2 μmol/g Hgb for the saline wash sample. At Day 1 the historical control level of 2,3-DPG was about 5 μmol/g Hgb. At Day 7 of storage, the 2,3-DPG levels in all the four samples continued to decline ranging from about 7 μmol/g Hgb for the 1:20 REJUVESOL dilution wash, about 6 μmol/g Hgb for the 1:40 REJUVESOL dilution wash, about 3 μmol/g Hgb 1:80 REJUVESOL dilution wash, to about 1 μmol/g Hgb for the saline wash sample. At Day 7 the historical control level of 2,3-DPG was about 3.5 μmol/g Hgb. At Day 14 of storage, the 2,3-DPG levels in all four samples declined again, ranging from about 5.5 μmol/g Hgb for the 1:20 REJUVESOL dilution wash, about 4 μmol/g Hgb for the 1:40 REJUVESOL dilution wash, to about 0.5 μmol/g Hgb for the 1:80 REJUVESOL dilution and the saline wash sample. At Day 14 the historical control level of 2,3-DPG was about 1 μmol/g Hgb. At Day 21 of storage, the 2,3-DPG levels for the 1:20 REJUVESOL dilution wash, the 1:40 REJUVESOL dilution wash, and the historical control continued to decline to about 4.5 μmol/g Hgb, 2 μmol/g Hgb, and 0.05 μmol/g Hgb respectively. The 2,3-DPG levels for the 1:80 REJUVESOL dilution wash and the saline wash sample were actually increased in comparison to the Day 14 measurement, and were at about 3 μmol/g Hgb and 1 μmol/g Hgb, respectively.

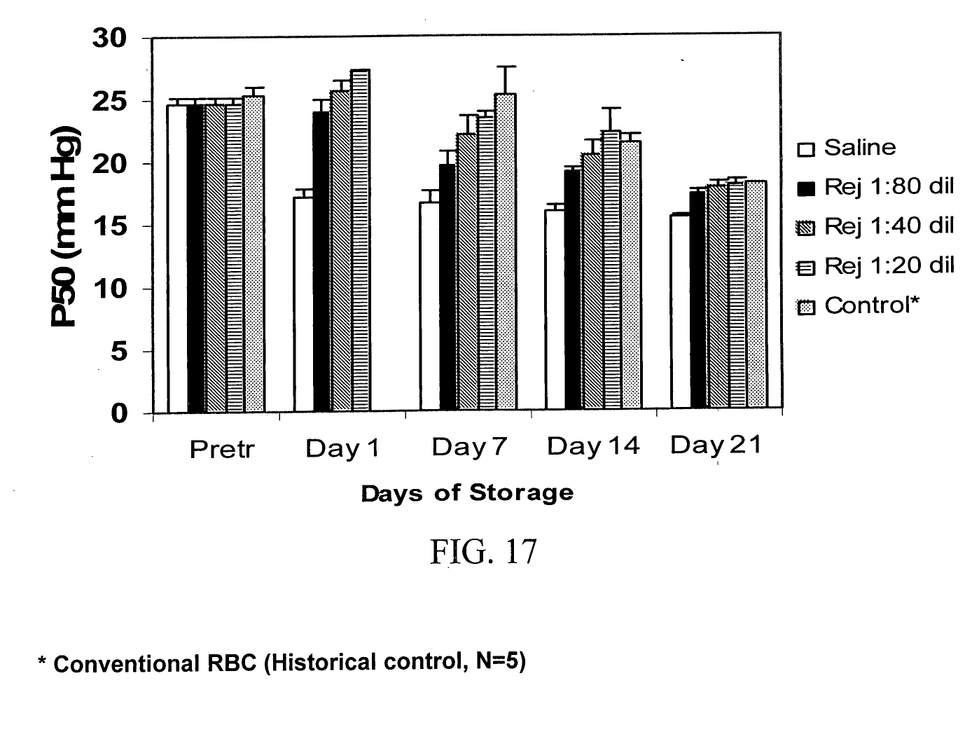

The cellular levels of P50 were also determined in all four samples following INACTINE™ treatment (saline wash, 1:80 REJUVESOL dilution wash, 1:40 REJUVESOL dilution wash, and 1:20 REJUVESOL dilution wash), and compared to a historical control of conventional RBCs over a period of 21 days of storage. The results of three independent experiments are shown in FIG. 17 (for the historical control the resuts represent five independent experiments). Before any treatment the P50 level in all of the samples was comparable, and it measured to be approximately 25 mm Hg. At Day 1 of storage, the P50 levels for the 1:20 and 1:40 REJUVESOL dilution washes were higher than at pretreatment, 27.5 mm Hg and 26 mm Hg, respectively. For the other samples the P50 levels declined ranging from 24 mm Hg to 17.5 mm Hg, for the 1:80 REJUVESOL dilution wash and the saline wash sample respectively. At Day 7 of storage, the P50 levels in all samples declined except for the historical control where the P50 was measured to be 26 mm Hg. The P50 levels in the other samples ranged from about 23 mm Hg for the 1:20 REJUVESOL dilution wash, about 22 mm Hg for the 1:40 REJUVESOL dilution wash, about 20 mm Hg for the 1:80 REJUVESOL dilution wash, to about 16 mm Hg for the saline wash sample. At Day 14 of storage, the P50 levels in all of the samples continued to decline ranging from about 22 mm Hg for the 1:20 REJUVESOL dilution wash, about 20 mm Hg for the 1:40 REJUVESOL dilution wash, to about 19 mm Hg for the 1:80 REJUVESOL dilution wash, and 16 mm Hg for the saline wash sample. At Day 14, P50 level for the historical control was about 21 mm Hg. At Day 21 of storage, the P50 levels in all of the samples continued to decline ranging from about 17.5 mm Hg for the 1:20 REJUVESOL dilution wash, the 1:40 REJUVESOL dilution wash, the 1:80 REJUVESOL dilution wash, and the historical control, to about 16 mm Hg for the saline wash sample.

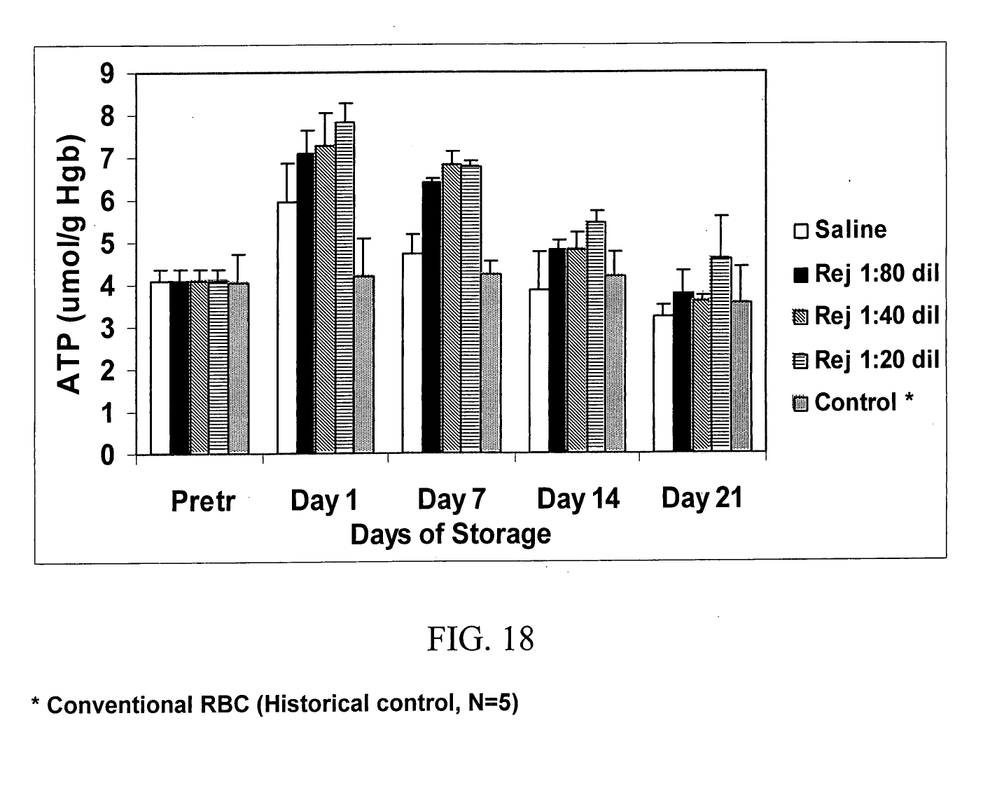

The cellular levels of ATP were determined in all four samples following INACTINE™ treatment (saline wash, 1:80 REJUVESOL dilution wash, 1:40 REJUVESOL dilution wash, and 1:20 REJUVESOL dilution wash), and compared to a historical control of conventional RBCs over a period of 21 days of storage. The results of three independent experiments are shown in FIG. 18 (for the historical control the resuts represent five independent experiments). Before any treatment the ATP levels was comparable in all of the samples, including the historical control and measured to be approximately 4 μmol/g Hgb. At Day 1 of storage, the ATP levels for all of the INACTINE™ samples increased in comparison to the pretreatment and the historical control, where the ATP levels were constant at about 4 μmol/g Hgb for Day 1 and all of the other time points measured (Day 7, Day 14, and Day 21). At Day 1 the ATP levels were about 7.9 μmol/g Hgb for the 1:20 REJUVESOL dilution wash, 7.2 μmol/g Hgb for the 1:40 REJUVESOL dilution wash, 7.0 μmol/g Hgb for the 1:80 REJUVESOL dilution wash and 5.9 μmol/g Hgb for the saline wash samples, respectively. At Day 7 of storage, the ATP levels in all samples declined and ranged from about 7 μmol/g Hgb for the 1:20 and 1:40 REJUVESOL dilution washes, about 6.2 μmol/g Hgb for the 1:80 REJUVESOL dilution wash, to about 4.5 μmol/g Hgb for the saline wash sample. At Day 14 of storage, the ATP levels in all of the samples continued to decline ranging from about 5.2 μmol/g Hgb for the 1:20 REJUVESOL dilution wash, about 5 μmol/g Hgb for the 1:40 and 1:80 REJUVESOL dilution washes, and 3.8 μmol/g Hgb for the saline wash sample. At Day 21 of storage, the ATP levels in all of the samples further declined to levels ranging from about 4.2 μmol/g Hgb for the 1:20 REJUVESOL dilution wash, about 3.8 μmol/g Hgb for the 1:40 and 1:80 REJUVESOL dilution washes, and about 3 μmol/g Hgb for the saline wash sample.

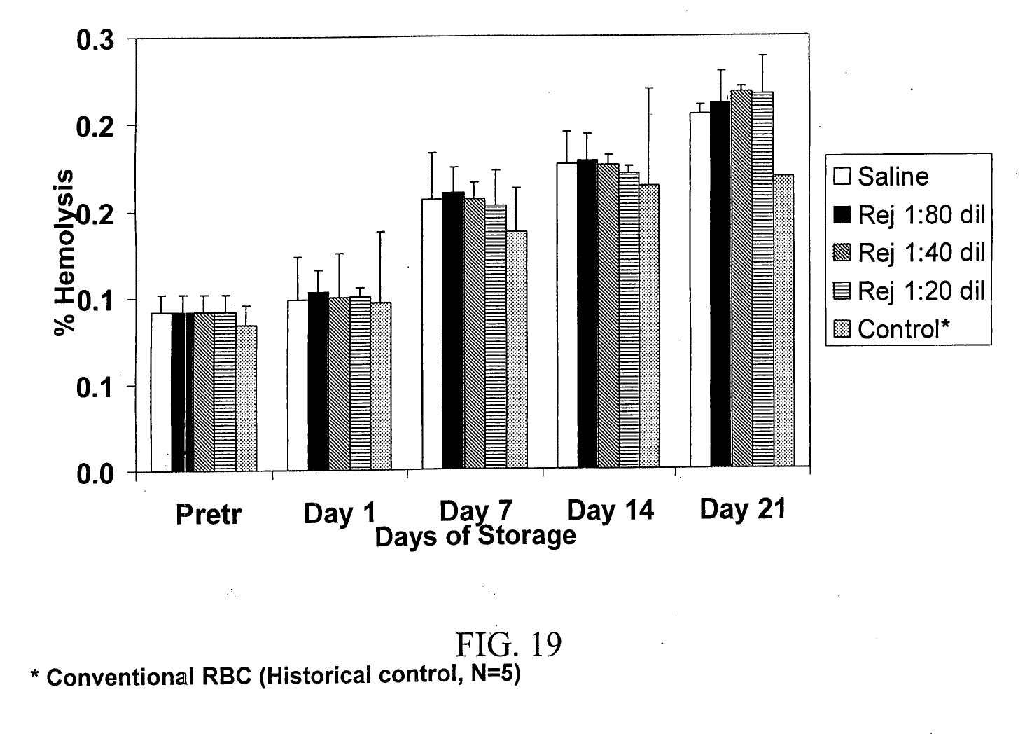

In addition to the biochemical functions, the percentage hemolysis of RBCs was determined in all four samples following INACTINE™ treatment (saline wash, 1:80 REJUVESOL dilution wash, 1:40 REJUVESOL dilution wash, and 1:20 REJUVESOL dilution wash), and compared to a historical control of conventional RBCs over a period of 21 days of storage. The results of three independent experiments are summarized in FIG. 19 (for the historical control the resuts represent five independent experiments). Following pretreatment all of the samples showed compable levels of hemolysis about 14%, and the historical control also showed a similar level of hemolysis, about 13%. At Day 1 of storage the hemolysis levels did not significantly increase in comparison to the pretreatment or the historical control levels and were measured to be about 15% for all samples. At Day 7, the levels of hemolysis increased for all samples, and they ranged from 21% for the 1:80 REJUVESOL dilution wash, to about 19% for the control sample. At Day 14, the hemolysis levels continued to increase for all samples, and they also continued to be very similar for the different REJUVESOL dilutions tested, approximately 22%. The historical control was also similar in value showing about 21% hemolysis. At Day 21, the highest levels of hemolysis were measured, ranging from 27% for the 1:20 and 1:40 REJUVESOL dilution washes to about 25 for the 1:80 REJUVESOL dilution and saline wash. At the Day 21 time point, the historical control showed about a 23% level of hemolysis.

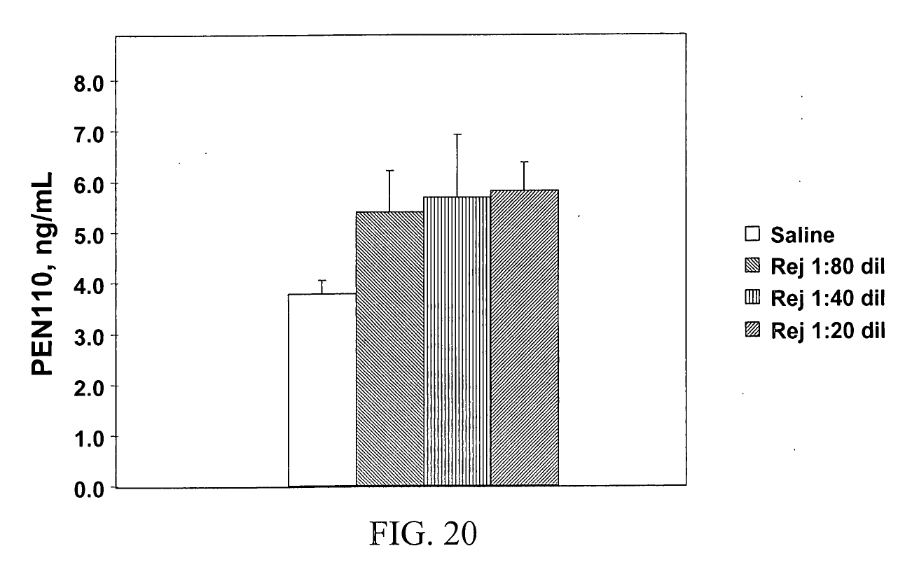

In addition to biochemical functions and hemolysis of RBCs, the effects of the INACTINE™ process and rejuvenation washes on the PEN110 removal from the treated samples were also examined. FIG. 20 shows measurements of PEN110 content as ng/ml in the four samples tested (saline wash, 1:80 REJUVESOL dilution wash, 1:40 REJUVESOL dilution wash, and 1:20 REJUVESOL dilution wash) as a summary of three independent experiments. As shown in FIG. 19, the saline wash sample showed the lowest PEN110 content, approximately 4 ng/ml of PEN110 following wash and removal. All of the samples treated with the various REJUVESOL dilution washes, showed comparable levels of PEN110 of about 5 to 5.5 ng/ml.

Taken together, the data presented in this example demonstrated that normal levels of 2,3-DPG, P50 and ATP can be restored in INACTINE™ treated RBCs by washing with 4 L of saline solution containing 1:40 to 1:80 part of REJUVESOL. In addition, rejuvenation during the wash provides a unique opportunity to neutralize biochemical consequences of PEN 110 treatment of RBCs.

Equivalents

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

1. A method for enhancing the function of a biological sample comprising contacting the biological sample with a solution comprising pyruvate, inosine, adenine, phosphate and an aziridino compound.

2. A method for enhancing the function of a biological sample comprising contacting the sample with a solution comprising pyruvate, inosine, adenine, and phosphate, then contacting the sample with an effective amount of an aziridino compound.

3. A method for enhancing the function of a biological sample comprising contacting the sample with an effective amount of an aziridino compound, then contacting the sample with a solution comprising pyruvate, inosine, adenine, and phosphate.

4. The method of claim 3, wherein the biological sample contains cells and wherein the cell-containing sample is contacted with pyruvate, inosine, adenine and phosphate during a step of cell washing.

5. The method of any of claims 1-3, wherein the pyruvate is present in the solution at a concentration of about 0.4 grams/liter to about 40 grams/liter.

6. The method of any of claims 1-3, wherein the inosine is present in the solution at a concentration of about 1 grams/liter to about 100 grams/liter.

7. The method of any of claims 1-3, wherein the adenine is present in the solution at a concentration of about 0.027 grams/liter to about 2.7 grams/liter.

8. The method of any of claims 1-3, wherein the phosphate is present as a dibasic phosphate at a concentration of about 0.4 grams/liter to about 40 grams/liter.

9. The method of any of claims 1-3, wherein the phosphate is present as a monobasic phosphate at a concentration of about 0.16 grams/liter to about 16 grams/liter.

10. The method of any of claims 1-3, wherein the aziridino compound is present at a concentration of about 0.01 mM to about 100 mM.

11. The method of claim 1, wherein the pyruvate is present in the solution at a concentration of about 2.75 grams/liter to about 6.05 grams/liter, the inosine is present in the solution at a concentration of about 6.70 grams/liter to about 14.74 grams/liter, the adenine is present in the solution at a concentration of about 0.17 grams/liter to about 0.37 grams/liter, the phosphate is present as a dibasic phosphate at a concentration of about 2.5 grams/liter to about 5.5 grams/liter and a monobasic phosphate at a concentration of about 1.0 grams/liter to about 2.2 grams/liter and the aziridino compound is present at a concentration of about 2.5 mM to about 55.0 mM.

12-13. (canceled)

14. The method of any of claims 1-3, wherein the biological sample comprises one or more red blood cells.

15. The method of any of claims 1-3, wherein the aziridino compound contains a linear alkyl group.

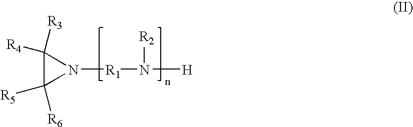

16-19. (canceled)

20. The method of claim 15, wherein the aziridino compound is an ethyleneimine oligomer.

21-23. (canceled)

24. The method of claim 14, wherein the pyruvate, inosine, phosphate, adenine and aziridino compound increase the amount of 2,3-DPG levels in the red blood cells relative to the amount of 2,3-DPG in red blood cells not contacted with the pyruvate, inosine, adenine, phosphate and aziridino compound.

25-26. (canceled)

27. The method of claim 14, wherein the pyruvate, inosine, phosphate, adenine and aziridino compound increase the amount of ATP in the red blood cells relative to the amount of ATP in red blood cells not contacted with the pyruvate, inosine, adenine phosphate, and aziridino compound.

28-29. (canceled)

30. The method of claim 24, wherein the pyruvate, inosine, phosphate, adenine and aziridino compound increase the P50 level in the red blood cells relative to the P50 level in red blood cells not contacted with the pyruvate, inosine, phosphate, adenine and aziridino compound.

31-58. (canceled)
